SEQUENCE LISTING (47 sequences)

SEQ ID NO: 1 - protein, 12 aa, Artificial Sequence
  Cardiac Targeting Peptide
APWHLSSQYSRT

SEQ ID NO: 2 - protein, 12 aa, Artificial Sequence
  Artificial Sequence for Cardiac Targeting Peptide
AAWHLSSQYSRT

SEQ ID NO: 3 - protein, 6 aa, Artificial Sequence
  Artificial Sequence for Cardiac Targeting Peptide
  misc_feature: Amiodarone is bound to the N-terminal amino acid
SQYSRT

SEQ ID NO: 4 - protein, 6 aa, Artificial Sequence
  Artificial Sequence for Cardiac Targeting Peptide
  misc_feature: Xaa (X) is any naturally occurring amino acid
  misc_feature (1)..(2): Xaa can be any naturally occurring amino acid
  misc_feature (4)..(5): Xaa can be any naturally occurring amino acid
XXYXXT

SEQ ID NO: 5 - protein, 6 aa, Artificial Sequence
  Artificial Sequence for Cardiac Targeting Peptide
SQYSRT

SEQ ID NO: 6 - protein, 6 aa, Artificial Sequence
  Artificial Sequence for Cardiac Targeting Peptide
  misc_feature: Xaa (X) is any naturally occurring amino acid
  misc_feature (3)..(3): Xaa can be any naturally occurring amino acid
  misc_feature (6)..(6): Xaa can be any naturally occurring amino acid
SQXSRX

SEQ ID NO: 7 - protein, 6 aa, Artificial Sequence
  Artificial Sequence for Cardiac Targeting Peptide
  misc_feature: Xaa (X) is any naturally occurring amino acid
  misc_feature (6)..(6): Xaa can be any naturally occurring amino acid
SQASRX

SEQ ID NO: 8 - protein, 6 aa, Artificial Sequence
  Artificial Sequence for Cardiac Targeting Peptide
  misc_feature: Xaa (X) is any naturally occurring amino acid
  misc_feature (6)..(6): Xaa can be any naturally occurring amino acid
SQWSRX

SEQ ID NO: 9 - protein, 6 aa, Artificial Sequence
  Artificial Sequence for Cardiac Targeting Peptide
  misc_feature: Xaa (X) is any naturally occurring amino acid
  misc_feature (6)..(6): Xaa can be any naturally occurring amino acid
SQYSRX

SEQ ID NO: 10 - protein, 6 aa, Artificial Sequence
  Artificial Sequence for Cardiac Targeting Peptide
SQASRT

SEQ ID NO: 11 - protein, 6 aa, Artificial Sequence
  Artificial Sequence for Cardiac Targeting Peptide
SQWRST

SEQ ID NO: 12 - protein, 6 aa, Artificial Sequence
  Artificial Sequence for Cardiac Targeting Peptide
  misc_feature: Xaa (X) is any naturally occurring amino acid
  misc_feature (2)..(2): Xaa can be any naturally occurring amino acid
  misc_feature (4)..(5): Xaa can be any naturally occurring amino acid
SXYXXT

SEQ ID NO: 13 - protein, 6 aa, Artificial Sequence
  Artificial Sequence for Cardiac Targeting Peptide
  misc_feature: Xaa (X) is any naturally occurring amino acid
  misc_feature (1)..(1): Xaa can be any naturally occurring amino acid
  misc_feature (4)..(5): Xaa can be any naturally occurring amino acid
XQYXXT

SEQ ID NO: 14 - protein, 6 aa, Artificial Sequence
  Artificial Sequence for Cardiac Targeting Peptide
  misc_feature: Xaa (X) is any naturally occurring amino acid
  misc_feature (1)..(2): Xaa can be any naturally occurring amino acid
  misc_feature (5)..(5): Xaa can be any naturally occurring amino acid
XXYSXT

SEQ ID NO: 15 - protein, 6 aa, Artificial Sequence
  Artificial Sequence for Cardiac Targeting Peptide
  misc_feature: Xaa (X) is any naturally occurring amino acid
  misc_feature (1)..(2): Xaa can be any naturally occurring amino acid
  misc_feature (4)..(4): Xaa can be any naturally occurring amino acid
XXYXRT

SEQ ID NO: 16 - protein, 6 aa, Artificial Sequence
  Artificial Sequence for Cardiac Targeting Peptide
  misc_feature: Xaa (X) is any naturally occurring amino acid
  misc_feature (4)..(5): Xaa can be any naturally occurring amino acid
SQYXXT

SEQ ID NO: 17 - protein, 6 aa, Artificial Sequence
  Artificial Sequence for Cardiac Targeting Peptide
  misc_feature: Xaa (X) is any naturally occurring amino acid
  misc_feature (2)..(2): Xaa can be any naturally occurring amino acid
  misc_feature (5)..(5): Xaa can be any naturally occurring amino acid
SXYSXT

SEQ ID NO: 18 - protein, 6 aa, Artificial Sequence
  Artificial Sequence for Cardiac Targeting Peptide
  misc_feature: Xaa (X) is any naturally occurring amino acid
  misc_feature (2)..(2): Xaa can be any naturally occurring amino acid
  misc_feature (4)..(4): Xaa can be any naturally occurring amino acid
SXYXRT

SEQ ID NO: 19 - protein, 6 aa, Artificial Sequence
  Artificial Sequence for Cardiac Targeting Peptide
  misc_feature: Xaa (X) is any naturally occurring amino acid
  misc_feature (1)..(1): Xaa can be any naturally occurring amino acid
  misc_feature (5)..(5): Xaa can be any naturally occurring amino acid
XQYSXT

SEQ ID NO: 20 - protein, 6 aa, Artificial Sequence
  Artificial Sequence for Cardiac Targeting Peptide
  misc_feature: Xaa (X) is any naturally occurring amino acid
  misc_feature (2)..(2): Xaa can be any naturally occurring amino acid
SXYSRT

SEQ ID NO: 21 - protein, 6 aa, Artificial Sequence
  Artificial Sequence for Cardiac Targeting Peptide
  misc_feature: Xaa (X) is any naturally occurring amino acid
  misc_feature (1)..(1): Xaa can be any naturally occurring amino acid
XQYSRT

SEQ ID NO: 22 - protein, 6 aa, Artificial Sequence
  Artificial Sequence for Cardiac Targeting Peptide
  misc_feature (4)..(4): Xaa can be any naturally occurring amino acid
SQYXRT

SEQ ID NO: 23 - protein, 6 aa, Artificial Sequence
  Artificial Sequence for Cardiac Targeting Peptide
  misc_feature: Xaa (X) is any naturally occurring amino acid
  misc_feature (1)..(2): Xaa can be any naturally occurring amino acid
  misc_feature (4)..(5): Xaa can be any naturally occurring amino acid
XXWXXT

SEQ ID NO: 24 - protein, 6 aa, Artificial Sequence
  Artificial Sequence for Cardiac Targeting Peptide
APWHLS

SEQ ID NO: 25 - protein, 12 aa, Artificial Sequence
  Artificial Sequence for Cardiac Targeting Peptide
AAWHLSSQYSRT

SEQ ID NO: 26 - protein, 12 aa, Artificial Sequence
  Artificial Sequence for Cardiac Targeting Peptide
APWALSSQYSRT

SEQ ID NO: 27 - protein, 12 aa, Artificial Sequence
  Artificial Sequence for Cardiac Targeting Peptide
APWHASSQYSRT

SEQ ID NO: 28 - protein, 12 aa, Artificial Sequence
  Artificial Sequence for Cardiac Targeting Peptide
APWHLASQYSRT

SEQ ID NO: 29 - protein, 12 aa, Artificial Sequence
  Artificial Sequence for Cardiac Targeting Peptide
APWHLSAQYSRT

SEQ ID NO: 30 - protein, 12 aa, Artificial Sequence
  Artificial Sequence for Cardiac Targeting Peptide
APWHLSSAYSRT

SEQ ID NO: 31 - protein, 12 aa, Artificial Sequence
  Artificial Sequence for Cardiac Targeting Peptide
APWHLSSQASRT

SEQ ID NO: 32 - protein, 12 aa, Artificial Sequence
  Artificial Sequence for Cardiac Targeting Peptide
APWHLSSQYART

SEQ ID NO: 33 - protein, 12 aa, Artificial Sequence
  Artificial Sequence for Cardiac Targeting Peptide
APWHLSSQYSAT

SEQ ID NO: 34 - protein, 12 aa, Artificial Sequence
  Artificial Sequence for Cardiac Targeting Peptide
APWHLSSQYSRA

SEQ ID NO: 35 - protein, 12 aa, Artificial Sequence
  Artificial Sequence for Cardiac Targeting Peptide
APAHLSSQYSRT

SEQ ID NO: 36 - protein, 8 aa, Artificial Sequence
  Artificial Sequence for Cardiac Targeting Peptide
HLSSQYSR

SEQ ID NO: 37 - protein, 11 aa, Artificial Sequence
  Artificial Sequence for Cardiac Targeting Peptide
APWHLSSQYSR

SEQ ID NO: 38 - protein, 11 aa, Artificial Sequence
  Artificial Sequence for Cardiac Targeting Peptide
PWHLSSQYSRT

SEQ ID NO: 39 - protein, 10 aa, Artificial Sequence
  Artificial Sequence for Cardiac Targeting Peptide
PWHLSSQYSR

SEQ ID NO: 40 - protein, 12 aa, Artificial Sequence
  Artificial Sequence for Cardiac Targeting Peptide
  misc_feature: X1 is W or Y
  misc_feature (3)..(3): Xaa can be any naturally occurring amino acid
APXHLSSQYSRT

SEQ ID NO: 41 - protein, 12 aa, Artificial Sequence
  Artificial Sequence for Cardiac Targeting Peptide
  misc_feature: X1 is W or Y
  misc_feature (9)..(9): Xaa can be any naturally occurring amino acid
APWHLSSQXSRT

SEQ ID NO: 42 - protein, 11 aa, Artificial Sequence
  Artificial Sequence for Cardiac Targeting Peptide
  misc_feature: X1 is W or Y
  misc_feature (2)..(2): Xaa can be any naturally occurring amino acid
PXHLSSQYSRT

SEQ ID NO: 43 - protein, 11 aa, Artificial Sequence
  Artificial Sequence for Cardiac Targeting Peptide
  misc_feature: X1 is W or Y
  misc_feature (8)..(8): Xaa can be any naturally occurring amino acid
PWHLSSQXSRT

SEQ ID NO: 44 - protein, 10 aa, Artificial Sequence
  Artificial Sequence for Cardiac Targeting Peptide
  misc_feature: X1 is W or Y
  misc_feature (1)..(1): Xaa can be any naturally occurring amino acid
XHLSSQYSRT

SEQ ID NO: 45 - protein, 10 aa, Artificial Sequence
  Artificial Sequence for Cardiac Targeting Peptide
  misc_feature: X1 is W or Y
  misc_feature (7)..(7): Xaa can be any naturally occurring amino acid
WHLSSQXSRT

SEQ ID NO: 46 - protein, 9 aa, Artificial Sequence
  Artificial Sequence for Cardiac Targeting Peptide
  misc_feature: X1 is W or Y
  misc_feature (1)..(1): Xaa can be any naturally occurring amino acid
XHLSSQYSR

SEQ ID NO: 47 - protein, 9 aa, Artificial Sequence
  Artificial Sequence for Cardiac Targeting Peptide
  misc_feature: X1 is W or Y
  misc_feature (7)..(7): Xaa can be any naturally occurring amino acid
WHLSSQXSR

(19) United States
(12) Patent Application Publication
(10) Pub. No.: US 2021/0085790 A1
(43) Pub. Date: Mar. 25, 2021

(54) IR700 Nanocompositions for Cardiac Therapies and Applications

(71) Applicant: Corralluma LLC., Houston, TX (US)

(72) Inventors: Thomas Hopkins, Houston, TX (US); Andrew Hopkins, Houston, TX (US)

(73) Assignee: Corralluma LLC, Houston, TX (US)

(21) Appl. No.: 16/846,174

(22) Filed: Apr. 10, 2020

Related U.S. Application Data
(60) Provisional application No. 62/832,260, filed on Apr. 10, 2019.

(51) Int. Cl. A61K 41/00; A61K 47/66; A61K 47/69

(52) U.S. Cl. CPC A61K 41/0071; B82Y 5/00; A61K 47/6929; A61K 47/66

(57) ABSTRACT

A nanocomposition for use in treating a cardiac condition using phthalocyanine dye, such as IR700. A nanocomposition having IR700, an 8PEG nanoparticle and a cardiac targeting peptide. Administering a product comprising IR700 to a patient, whereby the IR700 is delivered to cardiac tissue, and found in only cardiac tissue; and administering light to activate the IR700, thereby producing an ROS.

This application claims the right of priority to, and claims under 35 U.S.C. § 119(e)(1) the benefit of, U.S. provisional application Ser. No. 62/832,260 filed Apr. 10, 2019, the entire disclosure of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Apr. 9, 2020, is named CL-1a_Peptides_ST25.txt and is 16,038 bytes hi size.

### BACKGROUND OF THE INVENTION

### Field of the Invention

The present inventions relate generally to nanocompositions and uses of these compositions in dynamic therapies, imaging, diagnostics, theranostics and other applications.

The terms “nanocomposition”, “nanoparticle”, “nanomaterial”, “nanoparticle”, nanoproduct”, “nanoplatform”, “nanoconstruct”, “nanocomposite”, “nano”, and similar such terms, unless specified otherwise, are to be given their broadest possible meaning, and include particles, materials and compositions having a volumetric shape that has at least one dimension from about 1 nanometer (nm) to about 100 nm. Preferably, in embodiments, these volumetric shapes have their largest cross section from about 1 nm to about 100 nm.

The terms “nanocomposition”, “nanoconstructs”, “nanoplatform”, “nanocomposite”, and “nanoconstruct” and similar such terms, unless specified otherwise, are to be given their broadest possible meaning, and include a particle having a backbone material, e.g., a cage, support or matrix material, and one or more additives, e.g., agents, moieties, compositions, biologics, and molecules, that are associated with the backbone. Generally, the backbone material can be a nanoparticle. Generally, the additive is an active material having targeting, therapeutic, imaging, diagnostic, theranostic or other capabilities, and combinations and variations of these. In embodiments, the backbone material can be an active material, having targeting, therapeutic, imaging, diagnostic, theranostic or other capabilities, and combinations and variations of these. In embodiments both the additive and the backbone material are active materials. One, two, three or more different types of backbone materials, additives and combination and variations of these are contemplated.

The term “theranostic”, unless specified otherwise, is to be given its broadest possible meaning, and includes a particle, agent, composition, or material that has multiple capabilities and functions, including both imaging and therapeutic capabilities, both diagnostic and therapeutic capabilities, and combinations and variations of these and other features such as targeting.

The terms “imaging”, “imaging agent”, “imaging apparatus” and similar such terms, unless specified otherwise, should be given their broadest possible meaning, and would include apparatus, agents and materials that enhance, provide or enable the ability to detect, analyze and visualize the size, shape, position, composition, and combinations and variations of these as well as other features, of a structure, and in particular structures in animals, mammals and humans. Imaging agents would include contrast agents, dies, and similar types of materials. Examples of imaging apparatus and methodologies include: x-ray; magnetic resonance; computer axial tomography scan (CAT scan); proton emission tomography scan (PET scan); ultrasound; florescence; and, photo acoustic.

The term, “diagnostic”, unless specified otherwise, is to be given its broadest possible meaning, and would include identifying, determining, defining and combinations and variations of these, conditions, diseases and both, including conditions and diseases of animals, mammals and humans.

The term “therapeutic” and “therapy” and similar such terms, unless specified otherwise, are to be given their broadest possible meaning and would include addressing, treating, managing, mitigating, curing, preventing, and combinations and variations of these, conditions and diseases, including conditions and disease of animals, mammals and humans.

The terms “photodynamic therapy”, “PDT” and similar such terms, unless expressly stated otherwise, are to be given their broadest possible meaning and would include a method for ablating, (e.g., killing, destroying, rendering inert), biological tissue by photo-oxidation utilizing photosensitizer (“PS”) molecules. When the photosensitizer is exposed to a specific wavelength or wavelengths of light, it produces a form of oxygen from adjacent (e.g., in situ, local, intercellular, intracellular) oxygen sources, that kills nearby cells, e.g., reactive oxygen species (“ROS”), which includes any form of oxygen that are cyto-toxic to cells. It being understood that while light across all wavelengths, e.g., UV to visible to IR, is generally used as the activator of the PS, PS typically have a wavelength, or wavelengths where their absorption is highest.

The terms “activation dynamic therapy”, “dynamic therapy”, “dynamic therapy agent” and similar such terms, unless expressly stated otherwise, should be given their broadest possible meaning and would include PDT and PS, as well as agents that are triggered to product active oxygen, such as a reactive oxygen species (“ROS”) or other active therapeutic materials, when exposed to energy sources including energy sources other than light, as activators. These would include materials or agents that are activated by energy sources such as radio waves, other electromagnet radiation, magnetism, and sonic (e.g., Sonodynamic therapy or SDT).

The terms “photosensitizer” and “PS” and similar such terms, unless expressly stated otherwise, should be given their broadest possible meaning and would include any dye, molecule or modality that when exposed to light produces, or causes the production of ROS, or other active agents that are cyto-toxic to cells, kill tissue, ablates tissue, destroys tissue or renders a pathogen inert.

The terms “targeting agent” and “TA” and similar such terms, unless expressly stated otherwise, should be given their broadest possible meaning and would include any molecule, material or modality that is targeted to, or specific for, or capable of binding to or with, a predetermined cell type, receptor, or pathogen. TA would include, for example, a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations and variations of these.

As used herein, unless stated otherwise, room temperature is 25° C. And, standard ambient temperature and pressure is 25° C. and 1 atmosphere. Unless expressly stated otherwise all tests, test results, physical properties, and values that are temperature dependent, pressure dependent, or both, are provided at standard ambient temperature and pressure, this would include viscosities.

Generally, the term “about” and the symbol “˜” as used herein unless stated otherwise is meant to encompass a variance or range of ±10%, the experimental or instrument error associated with obtaining the stated value, and preferably the larger of these.

As used herein, unless specified otherwise, the recitation of ranges of values, a range, from about “x” to about “y”, and similar such terms and quantifications, serve as merely shorthand methods of referring individually to separate values within the range. Thus, they include each item, feature, value, amount or quantity falling within that range. As used herein, unless specified otherwise, each and all individual points within a range are incorporated into this specification, and are a part of this specification, as if they were individually recited herein.

As used herein, unless expressly stated otherwise terms such as “at least”, “greater than”, also mean “not less than”, i.e., such terms exclude lower values unless expressly stated otherwise.

This Background of the Invention section is intended to introduce various aspects of the art, which may be associated with embodiments of the present inventions. Thus, the forgoing discussion in this section provides a framework for better understanding the present inventions, and is not to be viewed as an admission of prior art.

### SUMMARY

There has been a long-standing and unfulfilled need for new and innovative drugs, medical products and imaging agents to address conditions of animals, mammals and humans. In particular, this long-standing and unfulfilled need is present in cardiology, including cardiac diagnoses and treatments.

The present inventions, among other things, solve these needs by providing the compositions, materials, articles of manufacture, devices, methods and processes taught, disclosed and claimed herein.

Thus, there is provided a nanocomposition having: a photosensitizer (PS), wherein the photosensitizer includes a phthalocyanine dye; a nanoparticle (NP); wherein the nanoparticle includes 8PEG; and, a targeting agent (TA), wherein the targeting agent includes a cardiac targeting peptide (CTP).

There is further provided these methods, treatments, compositions, kits, and nanocompositions having one or more of the following features: wherein the nanocomposition is configured to provide a photodynamic therapy for a cardiac indication; wherein the PS is IR700; the 8PEG is selected from the group constituting of 8PEGA and 8PEGMAL, and the CTP is one or more of SEQ ID NO: 1, SEQ ID 2, SEQ ID NO: 37 and SEQ ID NO: 38; wherein the PS is IR700; the 8PEG is selected from the group constituting of 8PEGA and 8PEGMAL, and the CTP is one or more of SEQ ID NO: 1 to SEQ ID 48; and, wherein the PS is IR700; the 8PEG is selected from the group constituting of 8PEGA and 8PEGMAL, and the CTP is one or more of SEQ ID NO: 1 to SEQ ID 48; and having 3 and less PS per NP.

Moreover, there is provided a nanocomposition, for use in treating a cardiac condition, the nanocomposition having: a photosensitizer (PS), wherein the photosensitizer is a phthalocyanine dye; a nanoparticle (NP); wherein the nanoparticle is selected from the group of 8PEG, 8PEGA and 8PEGMAL; and, a targeting agent (TA), wherein the targeting agent is a cardiac targeting peptide (CTP); wherein the nanocomposition is configured for providing a photodynamic therapy for the cardiac condition.

There is further provided these methods, treatments, compositions, kits, and nanocompositions having one or more of the following features: wherein the CTP includes one or more of SEQ ID NO: 1, SEQ ID 2, SEQ ID NO: 37 and SEQ ID NO: 38; wherein the CTP includes one or more of SEQ ID NO: 1 to SEQ ID 48; wherein the nanocomposition has less than 3 PS per NP; wherein the cardiac condition is an arrhythmia; wherein the cardiac condition is selected from the group consisting of atrial fibrillation, premature atrial contractions, wandering atrial pacemaker, multifocal atrial tachycardia, atrial flutter, supraventricular tachycardia, tachycardia, junctional rhythm, junctional tachycardia, premature junctional contraction, and premature ventricular contractions; wherein the cardiac condition is selected from the group consisting of accelerated idioventricular rhythm, monomorphic ventricular tachycardia, polymorphic ventricular tachycardia, ventricular fibrillation, heart blocks, long QT syndrome, and Brugada syndrome; and wherein the cardiac condition is selected from the group consisting of catecholaminergic polymorphic ventricular tachycardia, arrhythmogenic right ventricular dysplasia, and abnormal Purkinje potentials leading to ventricular arrhythmias including electrical storms.

Still further, there is provided a method of treating a cardiac condition, using any of these nanocomposition, the method including: administering to an animal a plurality of any of any of these nanocompositions; waiting a sufficient time for the nanocompositions to accumulate in a targeted cardiac tissue of the animal; and, illuminating the targeted cardiac tissue with light having a wavelength and sufficient energy to activate the PS, thereby producing reactive oxygen species (ROS).

There is further provided these methods, treatments, compositions, kits, and nanocompositions having one or more of the following features: wherein the light is a laser beam; wherein the illumination of the targeted cardiac tissue results in less than a 10 degree C. raise in temperature of the illuminated tissue; wherein the illumination of the targeted cardiac tissue results in less than a 5 degree C. raise in temperature of the illuminated tissue; wherein the illumination of the targeted cardiac tissue results in less than a 2 degree C. raise in temperature of the illuminated tissue; wherein the illumination of the targeted cardiac tissue does not raise the temperature of the illuminated tissue; wherein the illumination of the targeted cardiac tissue does not result in thermal breakdown of the illuminated tissue; and wherein the illumination of the targeted cardiac tissue does not result in induced optical breakdown.

Yet additionally, there is provided a kit having a container having a plurality of the nanocompositions of any of claims 1 to 5 and an illumination light source having a wavelength and power selected to activate the PS.

There is further provided these methods, treatments, compositions, kits, and nanocompositions having one or more of the following features: wherein the illumination light includes a disposable optical delivery device, wherein the optical delivery device can be an optical fiber; wherein the optical device can an LED; and wherein the optical delivery device can be an array of LEDs.

Furthermore, there is provided a composition for use in treating a cardiac condition using a photodynamic therapy, the composition having: a photosensitizer (PS), wherein the photosensitizer is a phthalocyanine dye; a core molecule; and, a targeting agent (TA), wherein the TA is specific to cardiac tissue.

There is further provided these methods, treatments, compositions, kits, and nanocompositions having one or more of the following features: wherein the composition is a nanocomposition and the core molecule is a nanoparticle NP; wherein the core molecule is selected from the group consisting of PEG, 8PEG, 8PEGA and 8PEGMAL; wherein the PS is water soluble; wherein the PS, TA and both are directly attached to the core molecule; wherein the direct attachment is a covalent bond; wherein the PS, TA and both are attached to the core by a linking moiety; wherein the TA is attached to the core by a linking moiety; wherein the TA is attached to the PS; wherein the TA is attached to the PS; and wherein the TA is not directly attached to the core; wherein the TA and PS form a conjugate, wherein the conjugate is attached to the core; wherein the core is an 8PEG nanoparticle, and the 8PEG nanoparticle has one free arm; wherein the core is an 8PEG nanoparticle, and the 8PEG nanoparticle has at least two free arms; wherein the core is an 8PEG nanoparticle, and the 8PEG nanoparticle has at least three free arms; wherein the core is an 8PEG nanoparticle, having no more than three PS; wherein the core is an 8PEG nanoparticle, having no more than two PS; wherein the core is an 8PEG nanoparticle, and a ratio of TA to PS is selected from the group consisting of and wherein the 2.5 to 1, 3 to 1, 4 to 1 and 5 to 1; wherein the core is an 8PEG nanoparticle, and wherein the composition has a hydrodynamic diameter selected from the group consisting of 70 nm and less, 50 nm and less, 25 nm and less, and 10 nm and less; and, wherein the core is an 8PEG nanoparticle, and wherein the nanoparticle has a mass selected from the group consisting of about 10 kDa and greater, about 20 kDa and greater, about 40 kDa and greater, and about 50 kDa and greater.

Yet further, there is provided a method of treating a cardiac condition having: administering to an animal a targeted nanoparticle having IR700; wherein the nanoparticle is a cardiac targeting agent; delivering light in the wavelength range of from about 600 nm to about 800 nm to a cardiac tissue having the target nanoparticle; whereby the IR700 is activated and the cardiac tissue is destroyed.

There is further provided these methods, treatments, compositions, kits, and nanocompositions having one or more of the following features: wherein the animal is a mammal; wherein the animal is a human; wherein the nanoparticle is 8PEGA; wherein the targeting agent is a cardiac specific protein; wherein the targeting agent targets cardiac muscle cells, and whereby only cardiac muscle cells are destroyed; wherein the targeting agent is a cardiac targeting peptide; wherein the targeting agent is one or more of SEQ ID NO: 1 to SEQ ID NO: 48; wherein the cardiac targeting agent is selected from the group consisting of: a Cardiac Targeting Peptides (CTP) having a net charge of between about +0.8 to +1.2 at pH=7, a CTP having a net charge of about +1.1 at pH=7, a CTP having an isoelectric point at between pH 9 and pH 9.5, a CTP having an isoelectric point at pH 9.35, a CTP having an average hydrophilicity index of between −0.2 and −0.6, a CTP having an average hydrophilicity index of −0.4, a CTP having (L) amino acids, and a CTP is having (D) amino acids.

Still further, there is provided treating a cardiac condition using IR700.

Yet additionally, there is provided administering a targeted nanocomposition to a patient, the nanocomposition having IR700, a CTP and an 8PEG nanoparticle, whereby the nanocomposition accumulated in a cardiact tissue of the patient.

Moreover, there is provided, administering a product having IR700 to a patient, whereby the IR700 is delivered to cardiac tissue, and found in only cardiac tissue; and administering light to activate the IR700, thereby producing an ROS.

Still further, there is provided a method of treating cardiac tissue, having: contacting an animal with a nanoparticle having a matrix, an active agent, and a cardiac targeting moiety; and administering an activator of said active agent to at least a portion of the cardiac tissue of said animal; wherein the active agent includes a phthalocyanine dye having a luminescent fluorophore moiety having at least one silicon containing aqueous-solubilizing moiety, wherein said phthalocyanine dye has a core atom selected from the group consisting of Si, Ge, Sn, and Al; wherein said phthalocyanine dye exists as a single core isomer, essentially free of other isomers; and has a reactive or activatable group.

Additionally, there is provided a method of treating cardiac tissue, having: contacting an animal with a nanoparticle having a matrix, an active agent, and a cardiac targeting moiety; and administering an activator of said active agent to at least a portion of the cardiac tissue of said animal; wherein the active agent consists essentially of a phthalocyanine dye having a luminescent fluorophore moiety having at least one silicon containing aqueous-solubilizing moiety, wherein said phthalocyanine dye has a core atom selected from the group consisting of Si, Ge, Sn, and Al; wherein said phthalocyanine dye exists as a single core isomer, essentially free of other isomers; and has a reactive or activatable group.

In addition, there is provided a method of treating cardiac tissue, having: contacting an animal with a nanoparticle having a matrix, an active agent, and a cardiac targeting moiety; and administering an activator of said active agent to at least a portion of the cardiac tissue of said animal; wherein the active agent consists of a phthalocyanine dye having a luminescent fluorophore moiety having at least one silicon containing aqueous-solubilizing moiety, wherein said phthalocyanine dye has a core atom selected from the group consisting of Si, Ge, Sn, and Al; wherein said phthalocyanine dye exists as a single core isomer, essentially free of other isomers; and has a reactive or activatable group.

There is further provided these methods, treatments, compositions, kits, and nanocompositions having one or more of the following features: wherein the matrix includes PEG, and wherein the said core atom of the dye is Si.

There is further provided these methods, treatments, compositions, kits, and nanocompositions having one or more of the following features: wherein the matrix includes PEG and wherein said dye has Formula I:

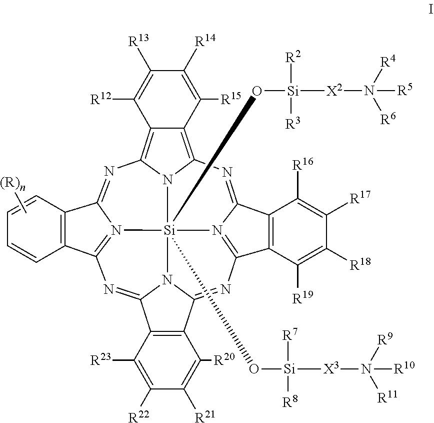

wherein:

R is a member selected from the group consisting of -L-Q and -L-Z¹;

L is a member selected from the group consisting of a direct link, or a covalent linkage, wherein said covalent linkage is linear or branched, cyclic or heterocyclic, saturated or unsaturated, having 1-60 atoms selected from the group consisting of C, N, P, O, and S, wherein L can have additional hydrogen atoms to fill valences, and wherein said linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds;

Q is a reactive or an activatable group;

Z¹ is a material;

n is 1 or 2;

R², R³, R⁷, and R⁸ are each independently selected from optionally substituted alkyl, and optionally substituted aryl;

R⁴, R⁵, R⁶, R⁹, R¹⁰, and R¹¹, if present, are each members independently selected from the group consisting of hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ includes a water soluble group;

R¹², R¹³, R¹⁴, R¹⁵, R¹⁶ R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring; and

X² and X³ are each members independently selected from the group consisting of C₁-C₁₀ alkylene optionally interrupted by a heteroatom, wherein if n is 1, the phthalocyanine may be substituted either at the 1 or 2 position and if n is 2, each R may be the same or different, or alternatively, they may join to form a 5- or 6-membered ring.

There is further provided these methods, treatments, compositions, kits, and nanocompositions having one or more of the following features: wherein the patient is a human; wherein the animal is a mammal; and, wherein the animal is a human; wherein the animal is a mammal.

### BRIEF DESCRIPTION OF THE DRAWINGS

FIG. 1 is a schematic formulaic representation of embodiments of targeted delivery nanocompositions, systems and products, in accordance with the present inventions.

FIG. 2 is a schematic formulaic representation of embodiments of various NP, TA and PS parings and combinations in accordance with the present inventions.

FIG. 3 is a formulaic representation of embodiments of linkers and functional group conversions in accordance with the present inventions.

FIG. 4 is a schematic formulaic representation of a nanocomposition in accordance with the present inventions.

FIG. 5A is a flow diagram of an embodiment of a process for making an embodiment of a nanocomposition in accordance with the present inventions.

FIG. 5B is a flow diagram of an embodiment of a process for making an embodiment of a nanocomposition in accordance with the present inventions.

FIG. 6A is a flow diagram of an embodiment of a process for making an embodiment of a PS for use in making a nanocomposition in accordance with the present inventions.

FIG. 6B is a flow diagram of an embodiment of a process for making an embodiment of a nanocomposition in accordance with the present inventions.

### DESCRIPTION OF THE PREFERRED EMBODIMENTS

The present inventions relate to the use of a photosensitizer, in compositions, including nanoparticle systems, having cardiac targeting agents, for use in photodynamic therapies, diagnostics and theranostics of animal, including mammal and human, cardiac conditions and tissues. The present inventions relate to the use of a preferred photosensitize, IR700, in compositions, including nanoparticle systems, having cardiac targeting agents, for use in photodynamic therapies, diagnostics and theranostics of animal, including human, cardiac conditions and tissues.

The present inventions further relate to nanocompositions. In particular, the present inventions provide nanocompositions for clinical (e.g., targeted therapeutic), diagnostic (e.g., imaging), and research applications in the field of cardiology.

Cardiac targeting proteins (“CTP”) are disclosed and taught in U.S. Pat. No. 9,249,184 and PCT patent application WO 2019/226785, the entire disclosures of each of which are incorporated herein by reference.

An embodiment of the present inventions is a composition having a core molecule, to which a cardiac specific TA and a PS are linked (e.g., chemically, covalently or otherwise attached). In preferred embodiments, the photosensitizer is a phthalocyanine dye, and the core molecule is a multi-arm nanoparticle, a linear molecule, PEG, a multi-arm PEG, 8PEG, 8PEGA and 8PEGMAL. These embodiments is used to provide cardiac PDT.

An embodiment of the present nanocompositions is a nanoparticle, a phthalocyanine PS, and a CTP TA. This embodiment is used to provide cardiac PDT.

An embodiment of the present nanocompositions is a nanoparticle, a phthalocyanine PS, where the phthalocyanine is a phthalocyanine die disclosed and taught in U.S. Pat. No. 7,005,518, and a CTP TA. This embodiment is used to provide cardiac PDT.

An embodiment of the present nanocompositions is a nanoparticle, a phthalocyanine PS, and a CTP TA, where the CTP is a CTP's disclosed and taught in U.S. Pat. No. 9,249,184 and PCT patent application WO 2019/226785. This embodiment is used to provide cardiac PDT.

An embodiment of the present nanocompositions is a nanoparticle, where the nanoparticle is PEG, and preferably 8PEGA, a phthalocyanine PS, and a CTP TA. This embodiment is used to provide cardiac PDT.

An embodiment of the present nanocompositions is a nanoparticle, where the nanoparticle is PEG, and preferably 8PEGA, a phthalocyanine PS, where the phthalocyanine is a phthalocyanine die disclosed and taught in U.S. Pat. No. 7,005,518, and a CTP TA. This embodiment is used to provide cardiac PDT.

An embodiment of the present nanocompositions is a nanoparticle, where the nanoparticle is PEG, and preferably 8PEGA, a phthalocyanine PS, where the phthalocyanine is a phthalocyanine die disclosed and taught in U.S. Pat. No. 7,005,518, and a CTP TA, where the CTP is a CTP's disclosed and taught in U.S. Pat. No. 9,249,184 and PCT patent application WO 2019/226785. This embodiment is used to provide cardiac PDT.

As used herein 8PEG refers to, and would include, any 8-arm polyethylene glycol (PEG) molecule (e.g., nanoparticle). 8PEG would include all 8PEGs where one or more of the end groups of the arms is modified. For example, 8PEG would include 8PEGA (8PEG-A, and similar terms) which is 8PEG having amine terminated end groups on the arms (one, two and preferably all arms). For example, 8PEG would include 8PEGMAL (8PEG-MAL and similar terms) which is 8PEG having maleimide terminated end groups on the arms (one, two and preferably all arms). These 8PEGs would include nanoparticles having a hydrodynamic diameter (e.g., size) of 25 nm and less, a hydrodynamic diameter of 10 nm and less, and having a hydrodynamic diameter of from about 30 nm to about 5 nm, and having a hydrodynamic diameter of from about 20 nm to about 5 nm. These 8PEGs would include nanoparticles that are 20 kilodaltons (kDa) and greater, that are 40 kDa and greater, and that are from about 15 kDa to about 50 kDa, and that are from about 5 kDa to about 100 kDa.

In an embodiment a nanoparticle having Dye IR700 and having a cardiac targeting protein of the 9,249,184 is used to provide a cardiac therapy.

In an embodiment a nanoparticle having Dye IR700 and having a cardiac targeting protein of the WO 2019/226785 is used to provide a cardiac therapy.

IRDye 700DX HHS Ester (“IR700”) is a preferred photosensitizer for the present embodiments of nanocompositions and for the treatment of cardiac conditions using the present embodiments of the targeted nanoparticle and nanocompositions based photodynamic therapies.

IR700 is a phthalocyanine dye that has minimal sensitive to photobleaching, and is thus preferred to many other organic fluorochromes. IR700 is water soluble, having good solubility. It is salt tolerant, having good salt tolerance. IR700 is available from LI-Cor and is an embodiment disclosed in U.S. Pat. No. 7,005,518, the entire disclosure of which is incorporated herein by reference.

IR700 has the following structure:

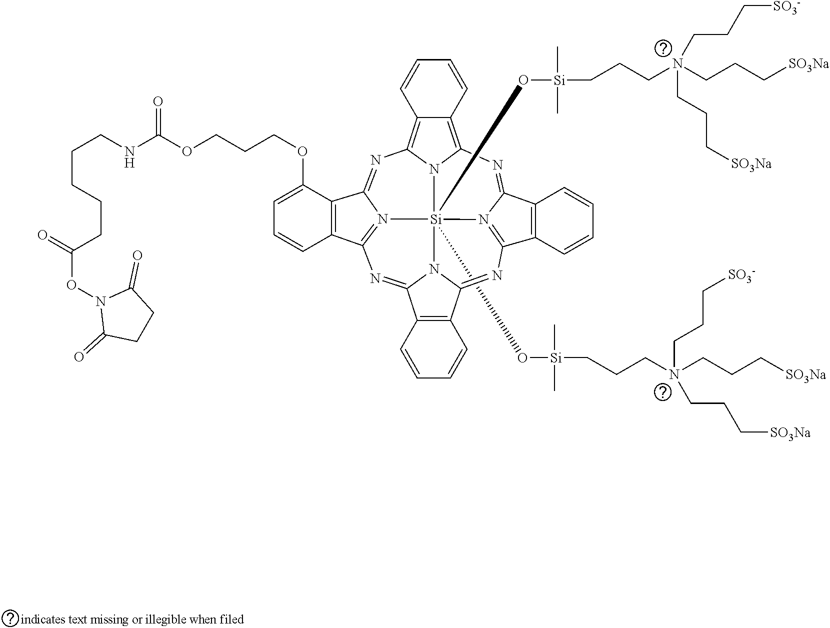

IR700 has the chemical formula C₇₄H₉₅N₁₂Na₄O₂₇S₆Si₃

IR700 has a molecular weight of 1954.21 g/mol.

IR700 has an exact mass of 1952.37

IR700 has a maximum absorbance of light at 689 nm. And, also shows much smaller absorbance peaks at 350 nm, and 625 nm.

In embodiments the cardiac targeted nanoparticle with IR700 is activated by delivering, to the cardiac tissue having this nanoparticle, light having a wavelength of from about 550 nm to about 750 nm, light having a wavelength of about 300 to 400, light having wavelengths of about 350 nm about 625 nm and about 689 nm, light from about 600 nm to about 800 nm, light from bout 650 nm to about 725 nm, light from about 675 nm to about 725 nm, light at bout 689 nm, light at 689 nm, and all wavelength within these ranges, as well as higher and lower wavelengths. In an embodiment the light is provided by a laser, and is a laser beam. Preferably, the power of the laser beam, and the amount of energy delivered to the cardiac tissue by the laser beam is below, and well below (e.g., at least 10% below, at least 20% below, at least 50% below) the threshold where the laser beam will heat, damage or cause laser induced optical breakdown. In a preferred embodiment the light that is delivered is eye safe.

Embodiments of the present nanaoconstructs provide improved methods of treating cardiac conditions and arrhythmias (e.g., atrial fibrillation, premature atrial contractions, wandering atrial pacemaker, multifocal atrial tachycardia, atrial flutter, supraventricular tachycardia, tachycardia, junctional rhythm, junctional tachycardia, premature junctional contraction, premature ventricular contractions, accelerated idioventricular rhythm, monomorphic ventricular tachycardia, polymorphic ventricular tachycardia, ventricular fibrillation, heart blocks, long QT syndrome, Brugada syndrome, catecholaminergic polymorphic ventricular tachycardia, and arrhythmogenic right ventricular dysplasia and abnormal Purkinje potentials leading to ventricular arrhythmias including electrical storms), using targeted therapies, including PDTs.

For example, in some embodiments, the present nanocompositions provide a method of treating (e.g., ablating) cardiac tissue, comprising: a) contacting an animal with a nano-particle comprising a matrix, a toxic (e.g., ablative) agent (e.g., photosensitizer), and a cardiac targeting moiety; and b) administering an activator of the toxic agent (e.g., light) to at least a portion of the cardiac tissue (e.g., heart) of the animal to activate the toxic agent. In some embodiments, administering the activator kills (e.g., ablates) cardiac tissue only where activator is administered and only to targeted cells. In some embodiments, the activator is light. In some embodiments, light from a laser (e.g., administered via open heart surgery or via a catheter or other mechanism). In some embodiments, the cardiac targeting moiety is a cardiac targeting peptide (e.g., SEQ ID NO: 36). In some embodiments, the photosensitizer is IR700. In some embodiments, the contacting is via intravenous administration. In some embodiments, the cardiac targeting moiety specifically targets cardiac myocytes. In some embodiments, the nanoparticle is a PEG molecule (e.g., 8-arm PEG). In some embodiments, the nanoparticle is approximately 10 nm or less in size.

In some embodiments, the animal is a human. For example, in some embodiments, the animal exhibits signs or symptoms of atrial fibrillation and the ablating reduces or eliminates the signs or symptoms.

In some embodiments, the method further comprises the step of imaging the nanoparticles in the animal. In some embodiments, the imaging is performed after the administering of activator and optionally determines a treatment course of action (e.g., further administering of activator, location of treatment and/or nanoparticles). In further embodiments, the present invention provides compositions and kits comprising the aforementioned nanoparticles and any additional components necessary, sufficient or useful in cardiac ablation and imaging.

In yet other embodiments, the present invention provides the use of the aforementioned nanoparticles (e.g., in cardiac ablation or treatment of cardiac arrhythmias). In still further embodiments, the present invention provides systems comprising a) the aforementioned nanoparticles; and b) an instrument for delivery of activator (e.g., a laser or ultrasound instrument). In some embodiments, systems further comprise imaging components (e.g., to image nanoparticles in cardiac tissue) and computer software and computer processor for controlling the system. In some embodiments, the computer software and computer processor are configured to control the delivery of the activator, image the nanoparticle, and displaying an image of the nanoparticle.

US Patent Publication No. 2015/0328315 teaches and disclose photodynamic therapies, nanocompositions, targeted nanocompositions, imaging and theranostics for cardiac related conditions and applications, the entire disclosure of which is incorporated herein by reference.

Table 1 provides examples of CTP for use as TAs in the present nanocompositions.

TABLE 1
Cardiac Targeting Peptides for use as
Targeting Agents in Nanocompositions

| ID Number | Sequence |
|---|---|
| (SEQ ID NO: 1) | APWHLSSQYSRT |
| (SEQ ID NO: 2) | AAWHLSSQYSRT (CTP-P2A) |
| (SEQ ID NO: 3) | Amiodarone-SQYSRT (CTP-B) |
| (SEQ ID NO: 4) | Xaa₁ Xaa₂ Y Xaa₃ Xaa₄ T |
| (SEQ ID NO: 5) | SQYSRT (CTP-B) |
| (SEQ ID NO: 6) | S Q Xaa₁ S R Xaa2 |
| (SEQ ID NO: 7) | SQASRXaa2 |
| (SEQ ID NO: 8) | SQWSRXaa2 |
| (SEQ ID NO: 9) | SQYSRXaa2 |
| (SEQ ID NO: 10) | SQASRT |
| (SEQ ID NO: 11) | SQWSRT |
| (SEQ ID NO: 12) | S Xaa2 Y Xaa3 Xaa 4 T |
| (SEQ ID NO: 13) | Xaa₁ Q Y Xaa3 Xaa4 T |
| (SEQ ID NO: 14) | Xaa₁ Xaa2 Y S Xaa₄ T |
| (SEQ ID NO: 15) | Xaa₁ Xaa2 Y Xaa3 R T |
| (SEQ ID NO: 16) | S Q Y Xaa3 Xaa₄ T |
| (SEQ ID NO: 17) | S Xaa2 Y S Xaa4 T |
| (SEQ ID NO: 18) | S Xaa2 Y Xaa3 R. T |
| (SEQ ID NO: 19) | Xaa₁ Q Y S Xaa₄ T |
| (SEQ ID NO: 20) | S Xaa2Y S RT |
| (SEQ ID NO: 21) | Xaa₁ Q Y S R T |
| (SEQ ID NO: 22) | S Q Y Xaa3 R T |
| (SEQ ID NO: 23) | Xaa₁ Xaa2 W Xaa3 Xaa₄ T |
| (SEQ ID NO: 24) | APWHLS (CTP-A) |
| (SEQ ID NO: 25) | Amiodarone-AAWHLSSQYSRT (CTP-P2A) |
| (SEQ ID NO: 26) | H4A |
| | APW ALSSQYSRT |
| (SEQ ID NO: 27) | L5A |
| | APWHASSQYSRT |
| (SEQ ID NO: 28) | S6A |
| | APWHLASQYSRT |
| (SEQ ID NO: 29) | S7A |
| | APWHLSAQYSRT |
| (SEQ ID NO: 30) | Q8A |
| | APWHLSSAYSRT |
| (SEQ ID NO: 31) | Y9A |
| | APWHLSSQASRT |
| (SEQ ID NO: 32) | S10A |
| | APWHLSSQYART |
| (SEQ ID NO: 33) | R11A |
| | APWHLSSQYSAT |
| (SEQ ID NO: 34) | T12A |
| | APWHLSSQYSRA |
| (SEQ ID NO: 35) | W3A |
| | APAHLSSQYSRT |
| (SEQ ID NO: 36) | APWHLSSQYSRT |
| (SEQ ID NO: 37) | HLSSQYSR |
| (SEQ ID NO: 38) | APWHLSSQYSR |
| (SEQ ID NO: 39) | PWHLSSQYSRT |
| (SEQ ID NO: 40) | PWHLSSQYSR |
| (SEQ ID NO: 41) | APX₁HLSSQYSRT where X₁ is W or Y |
| (SEQ ID NO: 42) | APWHLSSQX₁SRT where X₁ is W or Y |
| (SEQ ID NO: 43) | PX₁HLSSQYSRT where X₁ is W or Y |
| (SEQ ID NO: 44) | PWHLSSQX₁SRT where X₁ is W or Y |
| (SEQ ID NO: 45) | X₁HLSSQYSRT where X₁ is W or Y |
| (SEQ ID NO: 46) | WHLSSQX₁SRT where X₁ is W or Y |
| (SEQ ID NO: 47) | X₁HLSSQYSR where X₁ is W or Y |
| (SEQ ID NO: 48) | WHLSSQX₁SR where X₁ is W or Y |

SEQ ID NOS 1-35 are disclosed and taught in WO 2019/226785
SEQ ID NOS 36-48 are disclosed and taught in U.S. Pat. No. 9,249,184 (and correspond to sequence numbers 1-13 in that patent respectively)

The CTPs of Table 1 are further defined as follows. In an embodiment there is a twelve amino acid CTP (CTP12aa) having a sequence of Ala-Pro-Trp-His-Leu-Ser-Ser-Gln-Tyr-Ser-Arg-Thr (SEQ ID NO: 1). In an embodiment there is a six amino acid CTP (CTP6aa) having a sequence of SQYSRT (SEQ ID NO: 5), or a twelve amino acid CTP having a sequence of AAWHLSSQYSRT (SEQ ID NO: 2 (CTP-P2A)) In certain embodiments the sequence of Xaa₁ Xaa₂ Y Xaa₃ Xaa₄ T (SEQ ID NO: 4), in which Xaa₁, Xaa₂, Xaa₃, and Xaa₄ is any naturally occurring amino acid. In certain embodiments, Xaa₁ in the CTP₆ₐₐ of SEQ ID NO: 4 is serine (S). In certain embodiments, Xaa₂ in the CTP6aa of SEQ ID NO: 4 is glutamine (Q). In certain embodiments, Xaa₃ in the CTP6aa of SEQ ID NO: 4 is serine (S). In certain embodiments, Xaa₄ in the CTP6ₐₐ of SEQ ID NO: 1 is arginine (R). In certain embodiments, Xaa₁ and Xaa₂ in the CTP6aa of SEQ ID NO: 4 are serine (S) and glutamine (Q), respectively. In certain embodiments, Xaa₁ and Xaa3 in the CTP6aa of SEQ ID NO: 4 are both serine (S). In certain embodiments, Xaa₁ and Xaa4 in the CTP6aa of SEQ ID NO: 4 are serine (S) and arginine (R), respectively. In certain embodiments, Xaa₂ and Xaa₃ in the CTP6aa of SEQ ID NO: 4 are glutamine (Q) and serine (S), respectively. In certain embodiments, the CTP6aa comprises the sequence SQYSRT (SEQ ID NO: 5).

The CTPs of Table 1 are further defined as follows. In one aspect the CTP6aa comprises the sequence of S Q Xaa₁ S R Xaa2 (SEQ ID NO: 6). In certain embodiments, Xaa₁ in the CTP6aa of SEQ ID NO: 6 is alanine (A) and the CTP6aa comprises the sequence of SQASRXaa2 (SEQ ID NO: 7), or optionally, Xaa₁ in the CTP6aa of SEQ ID NO: 6 is tryptophan (W) and the CTP6aa comprises the sequence of SQWSRXaa2 (SEQ ID NO: 8), or Xaa₁ in the CTP6aa of SEQ ID NO: 6 is tyrosine (Y) and the CTP6aa comprises the sequence of SQYSRXaa2 (SEQ ID NO: 8). In certain embodiments, Xaa₂ in the CTP6aa of SEQ ID NO: 6 is threonine (T), and Xaa₁ in the CTP6aa of SEQ ID NO: 6 is alanine (A), tryptophan (W), or tyrosine (Y) comprising the sequence of SQASRT (SEQ ID NO: 10), SQWSRT (SEQ ID NO: 11), or SQYSRT (SEQ ID NO: 5), respectively. In certain embodiments, Xaa₂ in the CTP6aa of SEQ ID NO: 6 is alanine (A). In certain embodiments, Xaa₁ in the CTP6aa of SEQ ID NO: 6 is tyrosine (Y) and Xaa2 is alanine (A). In certain embodiments, the CTP6aa comprises the sequence SQYSRT (SEQ ID NO: 5).

The CTPs of Table 1 are further defined as follows. In certain embodiments, Xₐₐ₁ in the CTP6aa of SEQ ID NO: 4 is serine (S). In certain embodiments, Xaa2 in the CTP6aa of SEQ ID NO: 4 is glutamine (Q). In certain embodiments, Xaa3 in the CTP6aa of SEQ ID NO: 4 is serine (S). In certain embodiments, Xaa4 in the CTP6aa of SEQ ID NO: 4 is arginine (R). In certain embodiments, Xₐₐ₁ and Xaa2 in the CTP6ₐₐ of SEQ ID NO: 4 are serine (S) and glutamine (Q), respectively. In certain embodiments, Xaa₁ and Xaa3 in the CTP6aa of SEQ ID NO: 4 are both serine (S). In certain embodiments, Xₐₐ₁ and Xaa4 in the CTP6aa of SEQ ID NO: 4 are serine (S) and arginine (R), respectively. In certain embodiments, Xaa2 and Xaa3 in the CTP6aa of SEQ ID NO:4 are glutamine (Q) and serine (S), respectively. In certain embodiments, the CTP6aa comprises the sequence SQYSRT (SEQ ID NO: 5). In certain embodiments, a peptide comprising a CTP6aa comprising the sequence of Xaa₁ Xaa₂ W Xaa₃ Xaa₄ T (SEQ ID NO: 23), in which Xaa₁, Xaa₂, Xaa₃, and Xaa₄ is any naturally occurring amino acid. In certain embodiments, Xaa₁ in the CTP6aa of SEQ ID NO: 6 is alanine (A) and the CTP6aa comprises the sequence of SQASRXaa₂ (SEQ ID NO: 7), or optionally, Xaa₁ in the CTP6aa of SEQ ID NO: 6 is tryptophan (W) and the CTP6aa comprises the sequence of SQWSRXaa₂ (SEQ ID NO: 8), or Xaa₁ in the CTP6aa of SEQ ID NO: 6 is tyrosine (Y) and the CTP6aa comprises the sequence of SQYSRXaa₂ (SEQ ID NO: 9). In certain embodiments, Xaa₂ in the CTP6aa of SEQ ID NO: 6 is threonine (T), and Xₐₐ₁ in the CTP6aa of SEQ ID NO: 6 is alanine (A), tryptophan (W), or tyrosine (Y) comprising the sequence of SQASRT (SEQ ID NO: 10), SQWSRT (SEQ ID NO: 11), or SQYSRT (SEQ ID NO: 5), respectively. In certain embodiments, Xaa₂ in the CTP6aa of SEQ ID NO: 6 is alanine (A). In certain embodiments, Xaa₁ in the CTP6aa of SEQ ID NO: 6 is tyrosine (Y) and Xaa2 is alanine (A). In certain embodiments, the CTP6aa comprises the sequence SQYSRT (SEQ ID NO: 5).

In certain embodiments, a peptide comprising a CTP6aa of SEQ ID NO: 4 and SEQ ID NO: 6, for example SEQ ID NO: 5, is a recombinant or synthetically prepared peptide.

The CPT's target cardiac tissue, and in embodiments, particularly cardiac myocytes (cardiomyocytes). The CPT's, are linked to a nanoparticle to form a nanocomposition that also may have a PS. The CPT nanoparticle composition may be used for imaging. The CPT nanocomposition is transduced into cardiac tissue at much higher levels than it is transduced into other tissues, such as, for example, liver, kidney, lung, skeletal muscle, or brain. In certain embodiments the ratio of transduction of a CTP nanocomposition that into cardiac tissue relative to liver, kidney, lung, skeletal muscle or brain is at least 2:1, is at least 3:1 and greater.

Embodiments of the present nanocompositions, including 8PEG-CPT nanocompositions, have a PS that is a dye having the following formula of Formula I:

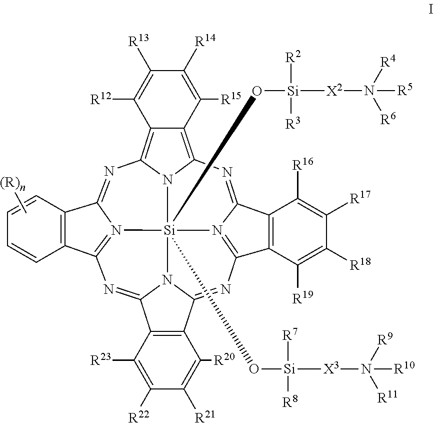

wherein: R is a member selected from the group consisting of -L-Q and -L-Z¹; L is a member selected from the group consisting of a direct link, or a covalent linkage, wherein said covalent linkage is linear or branched, cyclic or heterocyclic, saturated or unsaturated, having 1-60 atoms selected from the group consisting of C, N, P, O, and S, wherein L can have additional hydrogen atoms to fill valences, and wherein said linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds; Q is a reactive or an activatable group; Z¹ is a material; n is 1 or 2; R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; R¹², R¹³, R¹⁴, R¹⁵, R¹⁶ R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring; and X² and X³ are each members independently selected from the group consisting of C₁-C₁₀ alkylene optionally interrupted by a heteroatom, wherein if n is 1, the phthalocyanine may be substituted either at the 1 or 2 position and if n is 2, each R may be the same or different, or alternatively, they may join to form a 5- or 6-membered ring.

Embodiments of the present nanocompositions, including 8PEGA-CPT nanocompositions have a PS that is a dye having the following formula of Formula Ia:

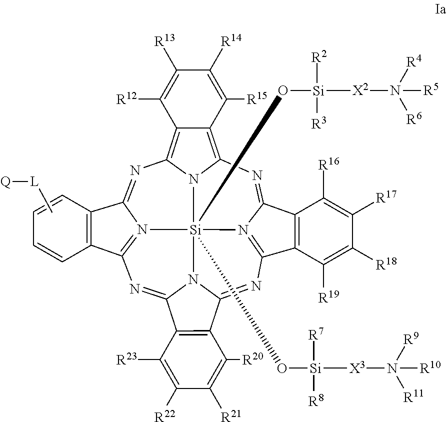

wherein: R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkanoyl, optionally substituted alkoxycarbonyl, optionally substituted alkylcarbamoyl, wherein at least one of R⁴, R⁵, R⁶, R⁹, R¹⁰, and R¹¹ comprises a water soluble group; and R¹², R¹³, R¹⁴, R¹⁵, R¹⁶ R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring.

In embodiments L has the following formula

—R¹—Y—X¹—Y¹—

wherein R¹ is a bivalent radical or a direct link; Y and Y¹ are each independently selected from the group consisting of a direct link, oxygen, an optionally substituted nitrogen and sulfur; and X¹ is a member selected from the group consisting of a direct link and C₁-C₁₀ alkylene optionally interrupted by a heteroatom.

In further embodiments, R¹ is a bivalent radical selected from the group consisting of optionally substituted alkylene, optionally substituted alkyleneoxycarbonyl, optionally substituted alkylenecarbamoyl, optionally substituted alkylenesulfonyl, optionally substituted alkylenesulfonylcarbamoyl, optionally substituted arylene, optionally substituted arylenesulfonyl, optionally substituted aryleneoxycarbonyl, optionally substituted arylenecarbamoyl, optionally substituted arylenesulfonylcarbamoyl, optionally substituted carboxyalkyl, optionally substituted carbamoyl, optionally substituted carbonyl, optionally substituted heteroarylene, optionally substituted heteroaryleneoxycarbonyl, optionally substituted heteroarylenecarbamoyl, optionally substituted heteroarylenesulfonylcarbamoyl, optionally substituted sulfonylcarbamoyl, optionally substituted thiocarbonyl, a optionally substituted sulfonyl, and optionally substituted sulfinyl.

In further embodiments, R¹ is R², R³, R⁷, and R⁸ are each independently selected from optionally substituted alkyl, and optionally substituted aryl, R⁴, R⁵, R⁶, R⁹, R¹⁰, and R¹¹, if present, are each members independently selected from an optionally substituted alkyl, wherein at least two members of the group consisting of R⁴, R⁵, R⁶, R⁷, R⁸, and R⁹ comprise a water soluble functional group; R¹², R¹³, R¹⁴, R¹⁵, R¹⁶ R¹⁷, R¹⁸, R¹⁹, R²⁰, R²¹, R²² and R²³ are each hydrogen, halogen, optionally substituted alkylthio, optionally substituted alkylamino and optionally substituted alkoxy, or in an alternative embodiment, at least one of R¹³, R¹⁴, and the carbons to which they are attached, or R¹⁷, R¹⁸, and the carbons to which they are attached, or R²¹, R²² and the carbons to which they are attached, join to form a fused benzene ring; X¹, X² and X³ are each members independently selected from the group consisting of C₁-C₁₀ alkylene optionally interrupted by a heteroatom; and Y and Y¹ are each independently selected from the group consisting of a direct link, oxygen, an optionally substituted nitrogen and sulfur.

In further embodiments, R², R³, R⁷, and R⁸ are each independently selected from optionally substituted methyl, ethyl, and isopropyl; R⁴, R⁵, R⁶, R⁹, R¹⁰, and R¹¹, if present, are each members independently selected from an optionally substituted alkyl, wherein at least two members of the group consisting of R⁴, R⁵, R⁶, R⁷, R⁸, and R⁹ comprise a substituent selected from the group consisting of a carboxylate (—CO₂⁻) group, a sulfonate (—SO₃⁻) group, a sulfonyl (—SO₂⁻) group, a sulfate (—SO₄⁻²) group, a hydroxyl (—OH) group, a phosphate (—OPO₃⁻²) group, a phosphonate (—PO₃⁻²) group, an amine (—NH₂) group and an optionally substituted quaternized nitrogen with each having an optional counter ion; R¹², R¹³, R¹⁴, R¹⁵, R¹⁶ R¹⁷, R¹⁸, R¹⁹, R²⁰, R²¹, R²² and R²³ are each hydrogen; X¹, X² and X³ are each members independently selected from the group consisting of C₁-C₁₀ alkylene optionally interrupted by a heteroatom; and Y and Y¹ are each independently selected from the group consisting of a direct link, oxygen, an optionally substituted nitrogen and sulfur.

Embodiments of the present nanocompositions, including 8PEGA-CPT nanocompositions have a PS that is a dye having the following formula:

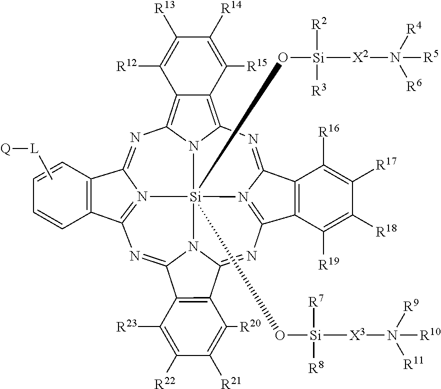

wherein Q is a reactive or an activatable group selected from the group consisting of an alcohol, an activated ester, an acyl halide, an alkyl halide, an optionally substituted amine, an anhydride, a carboxylic acid, a carbodiimide, hydroxyl, iodoacetamide, an isocyanate, an isothiocyanate, a maleimide, an NHS ester, a phosphoramidite, a platinum complex, a sulfonate ester, a thiol, and a thiocyanate.

Embodiments of the present nanocompositions, including 8PEGA-CPT nanocompositions have a PS that is a dye having the following formula of Formula 1b:

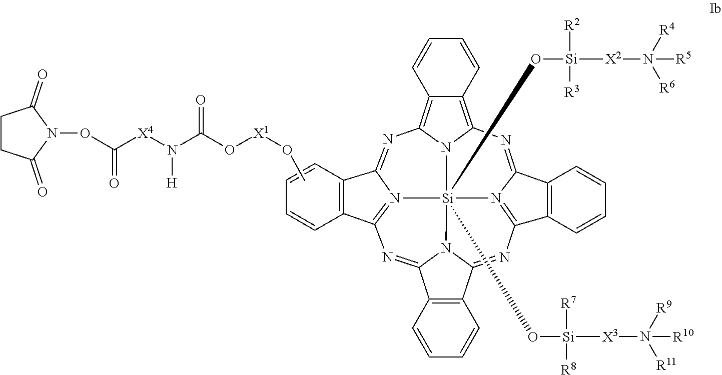

wherein X⁴ is a C₁-C₁₀ alkylene optionally interrupted by a heteroatom.

Embodiments of the present nanocompositions, including 8PEGA-CPT nanocompositions have a PS that is a dye having the formula of Formula 1c:

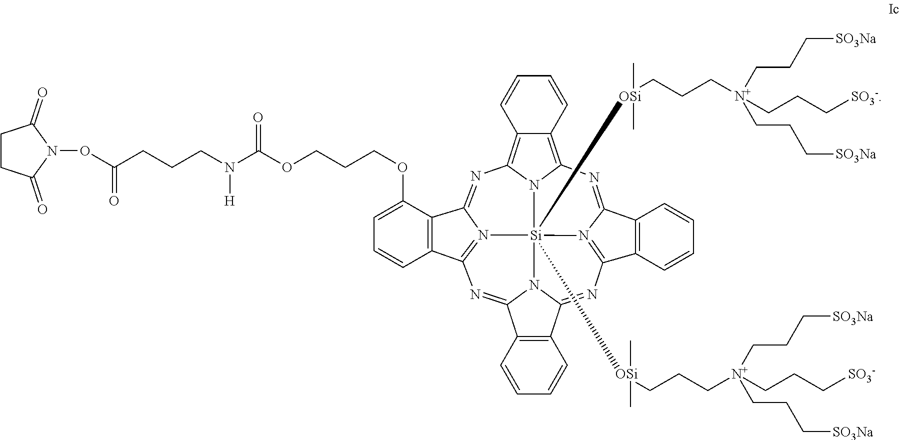

Embodiments of the present nanocompositions, including 8PEGA-CPT nanocompositions have a PS that is a dye having the following formula of Formula 1d:

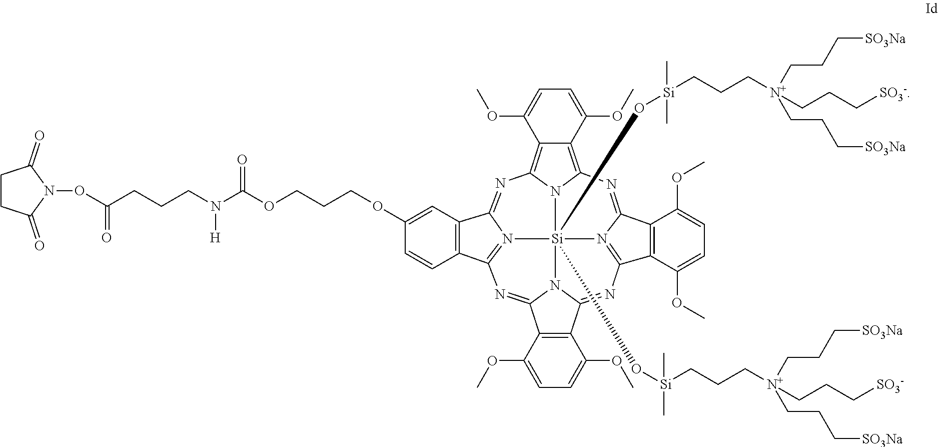

Embodiments of the present nanocompositions, including 8PEGA-CPT nanocompositions have a PS that is a dye having the following formula of Formula 1d-1:

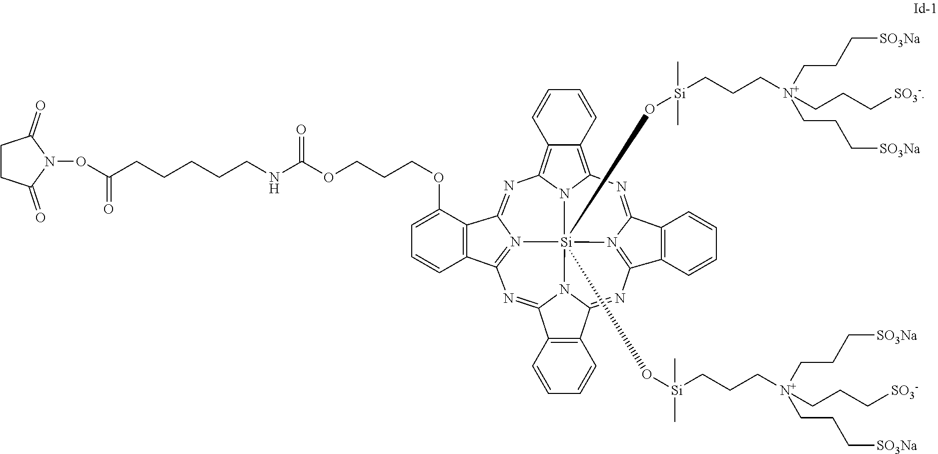

Embodiments of the present nanocompositions, including 8PEGA-CPT nanocompositions have a PS that is a dye having the following formula of Formula 1e:

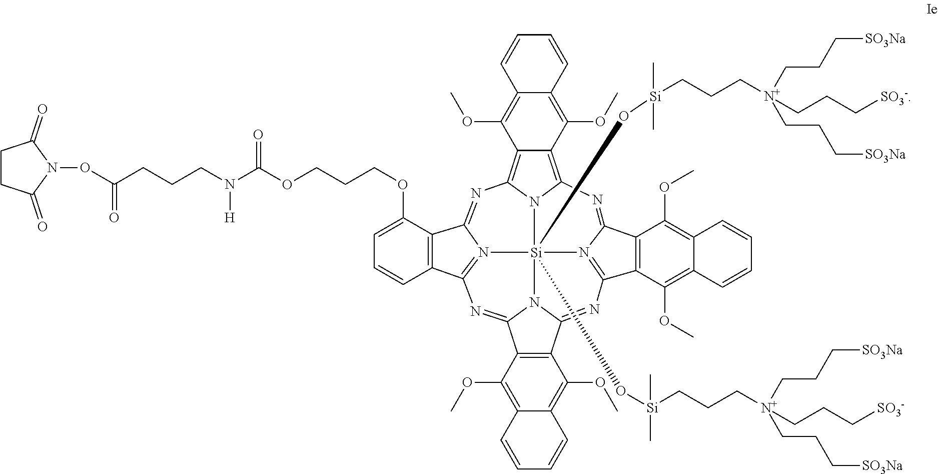

Embodiments of the present nanocompositions, including 8PEGA-CPT nanocompositions have a PS that is a dye having the following formula of Formula 1e-1:

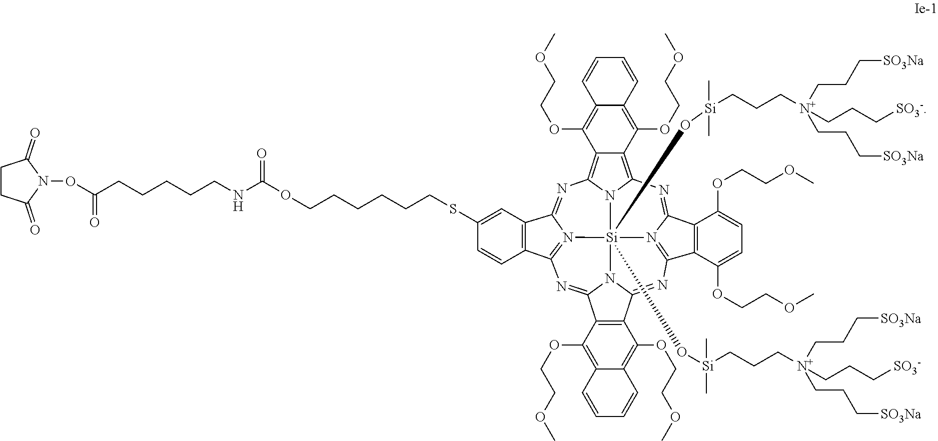

Embodiments of the present nanocompositions, including 8PEGA-CPT nanocompositions have a PS that is a dye having the following formula:

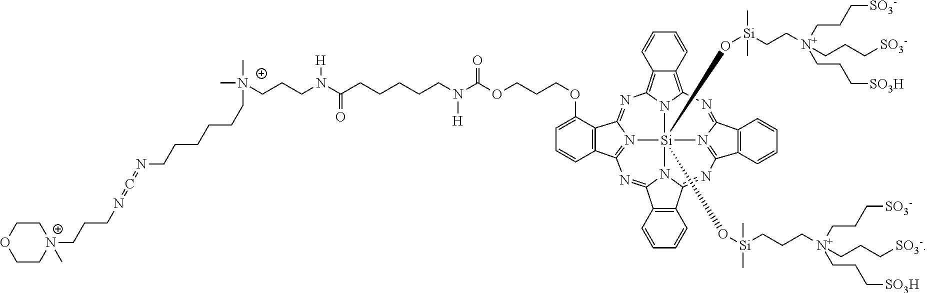

Embodiments of the present nanocompositions, including 8PEGA-CPT nanocompositions have a PS that is a dye having the following formula:

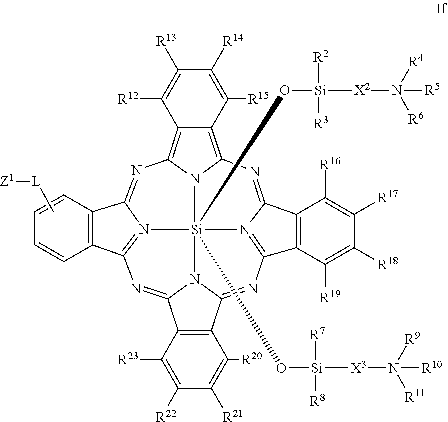

wherein: Z¹ is the nanoparticle; Lisa member selected from the group consisting of a direct link, or a covalent linkage, wherein said covalent linkage is linear or branched, cyclic or heterocyclic, saturated or unsaturated, having 1-60 atoms selected from the group consisting of C, N, P, O, and S, wherein L can have additional hydrogen atoms to fill valences, wherein said linkage contains any combination of ether, thiether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds; R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; R¹², R¹³, R¹⁴, R¹⁵, R¹⁶ R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring; and X² and X³ are each members independently selected from the group consisting of C₁-C₁ alkylene optionally interrupted by a heteroatom.

In general, and typically, PSs do not have any general affinity for specific tissues, other than certain classes generally favoring rapidly dividing cells (e.g. chlorins in cancer). Thus, in embodiments a targeted delivery of PDT, is beneficial, and in situations necessary to achieve a high contrast ratio between the target tissue, e.g., the tissue to be ablated and bystander tissues, e.g., the tissue that is intended to be unaffected by, and not damaged by, the PDT.

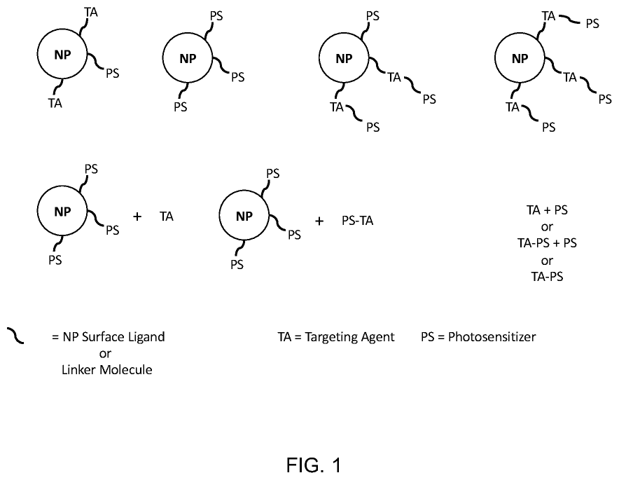

Targeted delivery of a PS may take several different forms: conjugation of a PS to a nanoparticle (NP), conjugation of a PS to a targeting agent (TA), conjugation of both a PS and TA to a NP (the PS being on the NP, the TA, or both), co-administration of a PS (with or without a NP) with a TA, or any combination thereof. Examples of some of these configurations for the present nanocompositions is shown in FIG. 1.

TAs include, for example, the CTPs of Table 1, a small molecule, a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations and variations of these.

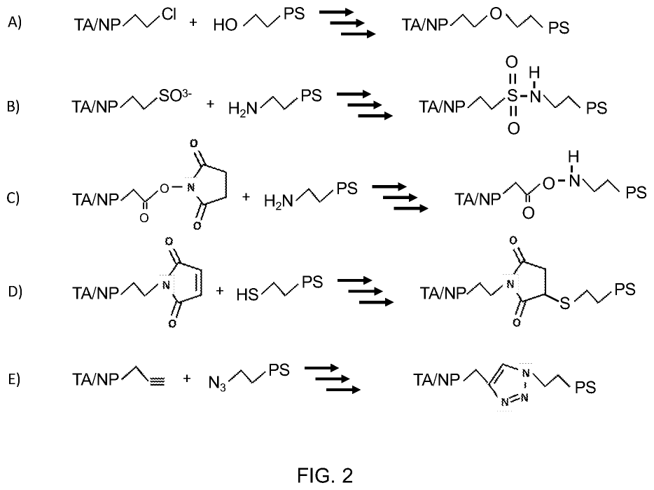

Turning to FIG. 2 there is shown embodiments of methods by which a PS may be covalently conjugated to a TA or NP. These methods are useful and applicable across most combinations, and so they are generally discussed as if they are a single method. Thus, any given method of NP conjugation should also be viable for TA conjugation. It further being understood that as a general requirement the functional groups employed should match each other. Tables 2-4 show a list of pairings and the resulting bonds formed between a TA, NP, or PS for examples of embodiments of combinations for embodiments of the present nanocompositions.

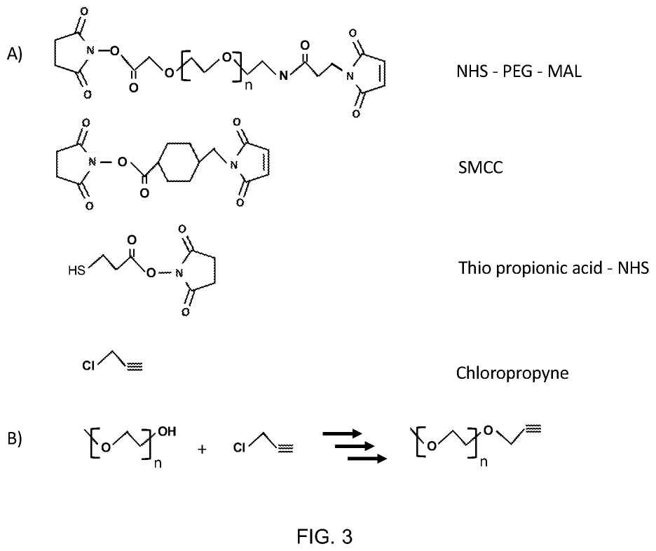

Optionally, conjugation of the PS to a TA, NP, or both, may include a spacer or linker molecule or group. Typically, this will not change the chemistry employed, but it can be used to convert functional groups from one set to another (e.g., an alcohol may be converted to an alkyne with a linking group to enable a different reaction protocol). The linkers may originate on the PS, TA, NP, or any combination, and may be a small molecule chain or polymer. FIG. 3 shows some example linkers and an end group conversion.

An embodiment of a final product would be a NP of small hydrodynamic diameter, preferably from a family of linear, branched, or cyclic macropolymers. Proteins, may also be used as they can be small enough, however, they may have competing pharma co-kinetic behavior with the TA. Examples of macropolymers for the NP would include: polyethylene glycol (PEG), poly amidoamine (PAMAM), polyethyleneimine (PEI), polyvinyl alcohol, and poly L-lysine. The preferred platform is PEG, specifically 8-arm branched PEG (8PEG), because of its widely known non-toxicity.

The various embodiments of the nanocompositions disclosed and taught herein can use or have multi-arm PEG NPs, this would include 8PEG and other numbers of arms, including 4-arm PEG, including 4PEGA (amine terminated end groups on the arms (one, two and preferably all arms)) and 4PEGMAL (having maleimide terminated end groups on the arms (one, two and preferably all arms)) and 6-arm PEG (including 6PEGA (amine terminated end groups on the arms (one, two and preferably all arms)) and 6PEGMAL (having maleimide terminated end groups on the arms (one, two and preferably all arms)).

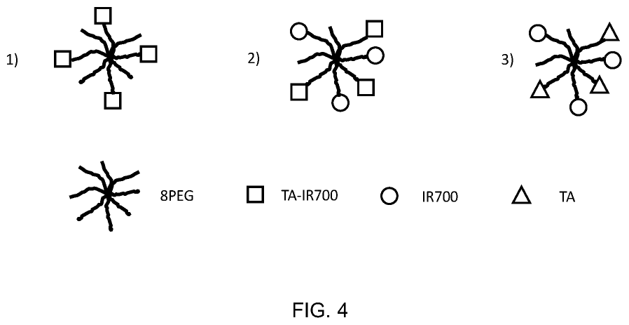

In an embodiment PEG, in particular 8PEG, conjugation can include both a TA and IR700 and may take, for example, the 3 Forms as shown in FIG. 4.

FIG. 4, Form 1) has a TA-IR700 conjugate that is attached to 8PEGA to provide a TA-PS-NP nanocomposition, having four IR700-TA conjugates attached to the 8PEGA.

FIG. 4, Form 2) is a TA-NP-TA-PS nanocomposition. Form 2) has three TA-IR700 conjugates attached to the 8PEGA, and has three IR700 dye molecules attached to the 8PEGA.

FIG. 4, Form 3) is a TA-NP-PA nanocomposition. Form 3) has three IR700 dye molecules attached to the 8PEGA, and has three TAs attached to the 8PEGA.

In an embodiment of Forms 1), 2) and 3) the TA is selected from one or more of the CTPs of Table 1. These forms do not have TAs and PSs bonded to every arm of the 8PEGA. Thus, Form 1) has three unbonded, or open, or non-active arms. Forms 2) and 3) have two unbonded, or open, or non-active arms. The unbonded arms, typically have end or terminus groups that are, for example, cysteine.

Additionally, the order of conjugation of a TA or IR700 to 8PEG is generally interchangeable for Forms 2) and 3); in this manner the IR700s can be attached first and then the TAs, or the TAs first and then the IR700s. A preferred embodiment would be Form 3), with the order of attachment being, attaching IR700s to 8PEG first, and then attaching the TAs to the 8PEGA. A benefit of this preferred method, among others, is to permit all 8PEGs to have at least one IR700 attached without risking the functionality of the TA by further modifying it.

Contrary to the general teaching of the art, it has been discovered that increasing the number of PS attached to the NP does not necessarily increase the amount of ROS produced, and does not necessarily increase the efficacy of the nanocomposition. Thus, for situations having four or more PS attached to an NP, and in particular 8PEGA, the ROS production and the efficacy of the nanocomposition may be decreased when compared to a nanocomposition having three or less PS. It is theorized that this occurs because of several facts relating to the spacing of the PS, and thus their ability to produce ROS from the in situ oxygen.

Thus, embodiments of IR700-8PEGA-CTP nanocompositions have from 1-2 IR700 dyes per 8PEGA, and 3-5 CTPs per 8PGEA. These and other embodiments can have a ratio of CTP to IR700 that is 2.5 to 1 and greater, 3 to 1 and greater, and 5 to 1 and greater. These and other embodiments can have 1, 2, 3, and 4 free arms and more. It being understood that embodiments having lower rations of CTP to IR700 per 8PEGA may also be utilized, including rations of 2 to 1 and 1 to 1. All combinations and variations of these configurations are also contemplated.

Thus, and generally, embodiments of PS-NP-TA nanocompositions have from 1-2 PS per 8PEGA, and 3-5 TA per 8PGEA. Embodiments of these, and other, nanocompositions have a ratio of TA to PS per NP that is 2.5 to 1 and greater, 3 to 1 and greater, and 5 to 1 and greater. These and other embodiments can have 1, 2, 3, and 4 free arms and more. It being understood that embodiments having lower rations of TA to PS per NP may also be utilized, including rations of 2 to 1 and 1 to 1. All combinations and variations of these configurations are also contemplated.

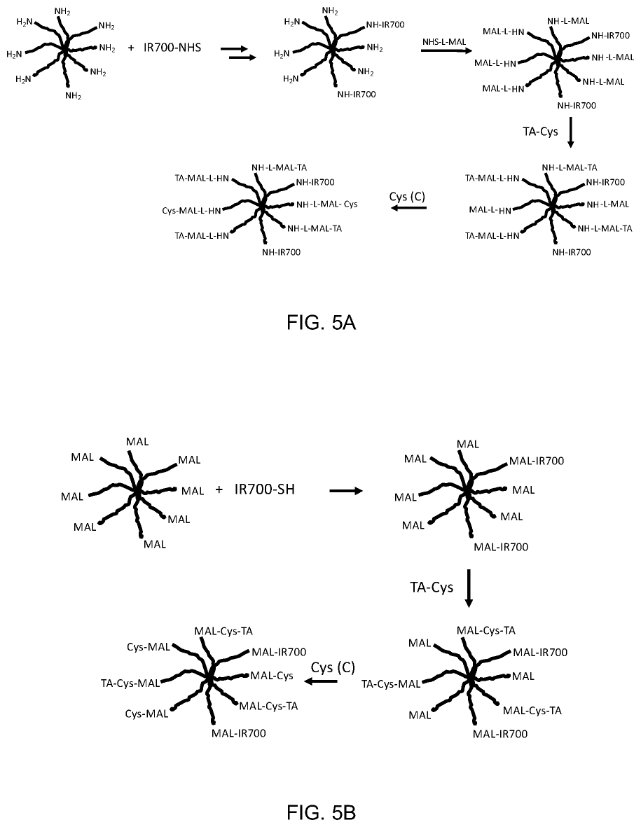

Turning to FIG. 5A there is provided an embodiment of a method to produce the nanocomposition of FIG. 4, Form 3).

FIG. 5A has the following steps:
IR700-NHS is added to 8PEG-Amine (8PEGA)
A linker (L) is added to 8PEGA to convert the amines to maleimides (MAL)
IR700-8PEGM is treated with thiol terminated (preferably cysteine, cys) TA
Additional free cysteine is added to cap unreacted MAL groups

Turning to FIG. 5B there is provided an embodiment of a method to produce the nanocomposition of FIG. 4, Form 3).

FIG. 5B has the following steps:
IR700-SH is added to 8PEGMAL
IR700-8PEGMAL is treated with thiol terminated TA (preferably cysteine, cys)
Additional free cysteine is added to cap unreacted MAL groups

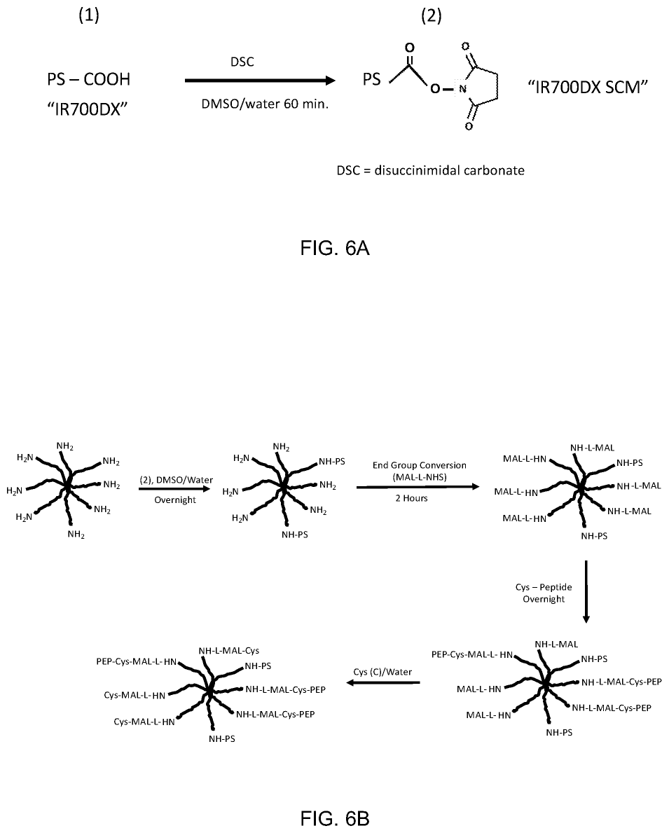

Turning to FIGS. 6A and 6B there is shown a general process for forming targeted nanocompositions for PDT, including an IR700-NP-CTP nanocomposition. “PEP”, (a peptide), is the TA, and can be a CTP of Table 1. The end group conversions step of FIG. 6B uses a chemical such as SMCC, BiPEG, or others, that converts the 8PEGA amines to maleimides (“MAL”).

FIG. 6A shows the preparation of the NHS ester (SCM, i.e., succinimidyl ester) for the PS, IR700 (formula (2)). FIG. 6B shows the preparation of the nanocomposition using the HHS ester (FIG. 6A, formula (2)) and a PEP TA, preferably a CTP from Table 1.

Covalent conjugation of a NP—X, PS-L-Q, or TA-Z in any combination may take many forms; generally the entities should have X, Q, and Z functional groups that are reactive towards each other. X, Q, and Z include, but are not limited to: alkyl halides, acyl halides, aromatic phenyls, aromatic halides (preferably iodo), carboxylic acids, sulfonic acids, phosphoric acids, alcohols (preferably primary), maleimides, esters, thiols, azides, aldehydes, alkenes (mono or diene), isocyanates, isothiocyanates, amines, anhydrides, or thiols. Tables 2-4 show the matching relevant combinations of NP—X, PS-L-Q, and TA-Z functional groups for conjugation.

TABLE 2
X and Q pairings of NP-X and PS-L-Q for
covalent conjugation [Makes PS(L)-NP-X]

| NP-X | PS-L-Q | Conditions | Covalent Bond |
|---|---|---|---|
| Alkyl Halide | PS-OH | Base, CHCl₃ or | Ether |
| (Chlorine) | PS-SH | DMSO | Thio Ether |
| | PS-COOH | | Ester |
| | PS-NH₂ | | |
| Acyl Halide | PS-NH₂ | 1.5:1 Base:PS-Y | Amide |
| (Chlorine) | PS-SH | (Opt) | Thio Ester |
| | PS-OH | CHCl₃ or DMSO | Ester |
| | PS-Phenyl | | Ketone |
| Aromatic (Phenyl) | PS-Cl | AlCl₃, CHCl₃ or | Alkyl chain |
| | PS-COCl | DMSO | ketone |
| Aromatic (Halide | PS-NH₂ | Base, CHCl₃ or | Secondary Amine |
| Phenyl) | PS-OH | DMSO | Ether |
| | PS-SH | | Thioether |
| Carboxylic Acid | PS-OH | Acid, CHCl₃ or | Ester |
| | PS-NH₂ | DMSO; | Amide |
| | PS-Cl | Acid, CHCl₃ or | Ester |
| | PS-SH | DMSO; | Thioester |
| | | Base, CHCl₃ or | |
| | | DMSO; | |
| | | Acid, CHCl₃ or | |
| | | DMSO | |
| Sulfonic Acid | PS-OH | 1.5:1 Base:PS-Y | Sulfonic ester |
| | PS-NH₂ | PCl₅, CHCl₃ or | Amino Sulfonate |
| | PS-SH | DMSO; | Sulfonic thioester |
| | | SOCl₂ may also be | |
| | | used | |
| Phosphoric Acid | PS-OH | 1.5:1 Base:PS-Y | Phosphoramidite |
| | PS-NH₂ | SOCl₂, CHCl₃ or | |
| | PS-SH | DMSO | |
| Alcohol (Primary) | PS-Cl PS-COOH | Base, CHCl₃ or | Ether |
| | PS-ester | DMSO; | Ester |
| | PS-thioester | Base, CHCl₃ or | Ester |
| | PS-anhydride | DMSO; | Ester |
| | PS-CHO | Base, CHCl₃ or | Ester |
| | PS-ITC | DMSO; | Ester |
| | PS-IC | Base, CHCl₃ or | Thiocarbamate |
| | | DMSO; | Urethane |
| | | Base, CHCl₃ or | |
| | | DMSO; | |
| | | Base, Pd catalyst, | |
| | | CHCl₃; | |
| | | 1.5:1 Base:PS-Y, | |
| | | CHCl₃; | |
| | | 1.5:1 Base:PS-Y, | |
| | | CHCl₃ | |
| Maleimide (MAL) | PS-SH | pH 6-8 in water; | Thioether |
| | | 1.5:1 Base:PS-Y in | |
| | | organic solvent | |
| Ester | PS-NH₂ | Acid, CHCl₃ or | Amide |
| | PS-OH | DMSO | Ester |
| | PS-SH | | Thioester |
| Thiol | PS-Mal | pH 6-8 in water; | Thioether |
| | PS-ITC | 1.5:1 Base:PS-Y, | Dithiocarbamate |
| | PS-IC | CHCl₃; | Thiourethane |
| | | 1.5:1 Base:PS-Y, | |
| | | CHCl₃ | |
| Azide | PS-Alkyne | Cu(I), CHCl₃ or | Triazole |
| | | DMSO; | |
| | | Cu free, CHCl₃ or | |
| | | water | |
| Aldehyde | PS-NH2 | CuI, TBHP, CHCl3; | Amide |
| | PS-OH | Base, Pd catalyst, | Ester |
| | | CHCl₃; | |
| Alkene | PS-Diene | Diels-Alder | Cyclo-alkyl |
| Alkyne | PS-Azide | Cu(I), CHCl₃ or | Triazole |
| | | DMSO; | |
| | | Cu free, CHCl₃ or | |
| | | water | |
| isocyanate | PS-OH | Base, CHCl₃; | Urethane |
| | PS-NH₂ | CHCl₃; | Urea |
| | PS-SH | Base, CHCl₃ | Thiourethane |
| isothiocyanate | PS-SH | 1.5:1 Base:PS-Y, | Dithiocarbamate |
| | PS-NH₂ | CHCl₃; | Thiourea |
| | PS-OH | pH 7.4 in water; | Thiocarbamate |
| | | 1.5:1 Base:PS-Y, | |
| | | CHCl₃ | |
| Amine (A) | PS-COOH | Acid, CHCl₃ or | Amide |
| | PS-COCl | DMSO; | Amide |
| | PS-NHS | Base (Opt), CHCl₃ | Amide |
| | PS-CHO | pH 7.4 in water; | Amide |
| | PS-ITC | Base, Pd catalyst, | Thiourea |
| | PS-IC | CHCl₃; | Urea |
| | | pH 7.4 in water; | |
| | | pH 7.4 in water | |
| Anhydride | PS-NH₂ | CHCl3 or DMSO; | Amide |
| | PS-OH | 1.5:1 Base:PS-Y, | Ester |
| | PS-SH | CHCl₃; | Thioester |
| | | 1.5:1 Base:PS-Y, | |
| | | CHCl₃ | |
| Thiol | PS-SH | Oxidant, CHCl₃ | Disulfide |

*Opt = optional;
NHS = N-hydroxy succinimide;
ITC = isothiocycanate;
IC = isocyanate

TABLE 3
X and Z pairings of PS(L)-NP-X or NP-X alone and TA-Z for covalent conjugation
[to make PS(L)-NP-TA the preferred material or NP-TA alone]

| PS(L)-NP-X (or NP- | | | |
|---|---|---|---|
| X) | TA-Z | Conditions | Covalent Bond |
| Alkyl Halide | TA-OH | Base, CHCl₃ or | Ether |
| (Chlorine) | TA-SH | DMSO | Thio Ether |
| | TA-COOH | | Ester |
| | TA-NH₂ | | |
| Acyl Halide | TA-NH₂ | 1.5:1 Base:PS-Y | Amide |
| (Chlorine) | TA-SH | (Opt) | Thio Ester |
| | TA-OH | CHCl₃ or DMSO | Ester |
| | TA-Phenyl | | Ketone |
| Aromatic (Phenyl) | TA-Cl | AlCl₃, CHCl₃ or | Alkyl chain |
| | TA-COCl | DMSO | ketone |
| Aromatic (Halide | TA-NH₂ | Base, CHCl₃ or | Secondary Amine |
| Phenyl) | TA-OH | DMSO | Ether |
| | TA-SH | | Thioether |
| Carboxylic Acid | TA-OH | Acid, CHCl₃ or | Ester |
| | TA-NH₂ | DMSO; | Amide |
| | TA-Cl | Acid, CHCl₃ or | Ester |
| | TA-SH | DMSO; | Thioester |
| | | Base, CHCl₃ or | |
| | | DMSO; | |
| | | Acid, CHCl₃ or | |
| | | DMSO | |
| Sulfonic Acid | TA-OH | 1.5:1 Base:PS-Y | Sulfonic ester |
| | TA-NH2 | PCl₅, CHCl₃ or | Amino Sulfonate |
| | TA-SH | DMSO; | Sulfonic thioester |
| | | SOCl₂ may also be | |
| | | used | |
| Phosphoric Acid | TA-OH | 1.5:1 Base:PS-Y | Phosphoramidite |
| | TA-NH₂ | SOCl₂, CHCl₃ or | |
| | TA-SH | DMSO | |
| Alcohol (Primary) | TA-Cl | Base, CHCl₃ or | Ether |
| | TA-COOH | DMSO; | Ester |
| | TA-ester | Base, CHCl₃ or | Ester |
| | TA-thioester | DMSO; | Ester |
| | TA-anhydride | Base, CHCl₃ or | Ester |
| | TA-CHO | DMSO; | Ester |
| | TA-ITC | Base, CHCl₃ or | Thiocarbamate |
| | TA-IC | DMSO; | Urethane |
| | | Base, CHCl₃ or | |
| | | DMSO; | |
| | | Base, Pd catalyst, | |
| | | CHCl₃; | |
| | | 1.5:1 Base:PS-Y, | |
| | | CHCl₃; | |
| | | 1.5:1 Base:PS-Y, | |
| | | CHCl₃ | |
| Maleimide (Mal) | TA-SH | pH 6-8 in water; | Thioether |
| | | 1.5:1 Base:PS-Y in | |
| | | organic solvent | |
| Ester | TA-NH₂ | Acid, CHCl₃ or | Amide |
| | TA-OH | DMSO | Ester |
| | TA-SH | | Thioester |
| Thiol | TA-Mal | pH 6-8 in water; | Thioether |
| | TA-ITC | 1.5:1 Base:PS-Y, | Dithiocarbamate |
| | TA-IC | CHCl₃; | Thiourethane |
| | | 1.5:1 Base:PS-Y, | |
| | | CHCl₃ | |
| Azide | TA-Alkyne | Cu(I), CHCl₃ or | Triazole |
| | | DMSO; | |
| | | Cu free, CHCl₃ or | |
| | | water | |
| Aldehyde | TA-NH2 | CuI, TBHP, CHCl3; | Amide |
| | TA-OH | Base, Pd catalyst, | Ester |
| | | CHCl₃; | |
| Alkene | TA-Diene | Diels-Alder | Cyclo-alkyl |
| Alkyne | TA-Azide | Cu(I), CHCl₃ or | Triazole |
| | | DMSO; | |
| | | Cu free, CHCl₃ or | |
| | | water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:PS-Y, | Dithiocarbamate |
| | TA-NH₂ | CHCl₃; | Thiourea |
| | TA-OH | pH 7.4 in water; | Thiocarbamate |
| | | 1.5:1 Base:PS-Y, | |
| | | CHCl₃ | |
| Amine (A) | TA-COOH | Acid, CHCl₃ or | Amide |
| | TA-COCl | DMSO; | Amide |
| | TA-NHS | Base (Opt), CHCl₃ | Amide |
| | TA-CHO | pH 7.4 in water; | Amide |
| | TA-ITC | Base, Pd catalyst, | Thiourea |
| | TA-IC | CHCl₃; | Urea |
| | | pH 7.4 in water; | |
| | | pH 7.4 in water | |
| Anhydride | TA-NH₂ | CHCl3 or DMSO; | Amide |
| | TA-OH | 1.5:1 Base:PS-Y, | Ester |
| | TA-SH | CHCl₃; | Thioester |
| | | 1.5:1 Base:PS-Y, | |
| | | CHCl₃ | |
| Thiol | TA-SH | Oxidant, CHCl₃ | Disulfide |

*Opt = optional;
NHS = N-hydroxy succinimide;
ITC = isothiocycanate;
IC = isocyanate

TABLE 4
Q and Z pairings of PS-L-Q and TA-Z for covalent conjugation [This
makes PS(L)-TA, that could potentially be used (no NP) or could then
be attached to the NP to form a new (and never tried) form PA-TS-NP]

| PS-L-Q | TA-Z | Conditions | Covalent Bond |
|---|---|---|---|
| Alkyl Halide | TA-OH | Base, CHCl₃ or | Ether |
| (Chlorine) | TA-SH | DMSO | Thio Ether |
| | TA-COOH | | Ester |
| | TA-NH₂ | | |
| Acyl Halide | TA-NH₂ | 1.5:1 Base:PS-Y | Amide |
| (Chlorine) | TA-SH | (Opt) | Thio Ester |
| | TA-OH | CHCl₃ or DMSO | Ester |
| | TA-Phenyl | | Ketone |
| Aromatic (Phenyl) | TA-Cl | AlCl₃, CHCl₃ or | Alkyl chain |
| | TA-COCl | DMSO | ketone |
| Aromatic (Halide | TA-NH₂ | Base, CHCl₃ or | Secondary Amine |
| Phenyl) | TA-OH | DMSO | Ether |
| | TA-SH | | Thioether |
| Carboxylic Acid | TA-OH | Acid, CHCl₃ or | Ester |
| | TA-NH₂ | DMSO; | Amide |
| | TA-Cl | Acid, CHCl₃ or | Ester |
| | TA-SH | DMSO; | Thioester |
| | | Base, CHCl₃ or | |
| | | DMSO; | |
| | | Acid, CHCl₃ or | |
| | | DMSO | |
| Sulfonic Acid | TA-OH | 1.5:1 Base:PS-Y | Sulfonic ester |
| | TA-NH₂ | PCl₅, CHCl₃ or | Amino Sulfonate |
| | TA-SH | DMSO; | Sulfonic thioester |
| | | SOCl₂ may also be | |
| | | used | |
| Phosphoric Acid | TA-OH | 1.5:1 Base:PS-Y | Phosphoramidite |
| | TA-NH₂ | SOCl₂, CHCl₃ or | |
| | TA-SH | DMSO | |
| Alcohol (Primary) | TA-Cl | Base, CHCl₃ or | Ether |
| | TA-COOH | DMSO; | Ester |
| | TA-ester | Base, CHCl₃ or | Ester |
| | TA-thioester | DMSO; | Ester |
| | TA-anhydride | Base, CHCl₃ or | Ester |
| | TA-CHO | DMSO; | Ester |
| | TA-ITC | Base, CHCl₃ or | Thiocarbamate |
| | TA-IC | DMSO; | Urethane |
| | | Base, CHCl₃ or | |
| | | DMSO; | |
| | | Base, Pd catalyst, | |
| | | CHCl₃; | |
| | | 1.5:1 Base:PS-Y, | |
| | | CHCl₃; | |
| | | 1.5:1 Base:PS-Y, | |
| | | CHCl₃ | |
| Maleimide (Mal) | TA-SH | pH 6-8 in water; | Thioether |
| | | 1.5:1 Base:PS-Y in | |
| | | organic solvent | |
| Ester | TA-NH₂ | Acid, CHCl₃ or | Amide |
| | TA-OH | DMSO | Ester |
| | TA-SH | | Thioester |
| Thiol | TA-Mal | pH 6-8 in water; | Thioether |
| | TA-ITC | 1.5:1 Base:PS-Y, | Dithiocarbamate |
| | TA-IC | CHCl₃; | Thiourethane |
| | | 1.5:1 Base:PS-Y, | |
| | | CHCl₃ | |
| Azide | TA-Alkyne | Cu(I), CHCl₃ or | Triazole |
| | | DMSO; | |
| | | Cu free, CHCl₃ or | |
| | | water | |
| Aldehyde | TA-NH2 | CuI, TBHP, CHCl3; | Amide |
| | TA-OH | Base, Pd catalyst, | Ester |
| | | CHCl₃; | |
| Alkene | TA-Diene | Diels-Alder | Cyclo-alkyl |
| Alkyne | TA-Azide | Cu(I), CHCl₃ or | Triazole |
| | | DMSO; | |
| | | Cu free, CHCl₃ or | |
| | | water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:PS-Y, | Dithiocarbamate |
| | TA-NH₂ | CHCl₃; | Thiourea |
| | TA-OH | pH 7.4 in water; | Thiocarbamate |
| | | 1.5:1 Base:PS-Y, | |
| | | CHCl₃ | |
| Amine (A) | TA-COOH | Acid, CHCl₃ or | Amide |
| | TA-COCl | DMSO; | Amide |
| | TA-NHS | Base (Opt), CHCl₃ | Amide |
| | TA-CHO | pH 7.4 in water; | Amide |
| | TA-ITC | Base, Pd catalyst, | Thiourea |
| | TA-IC | CHCl₃; | Urea |
| | | pH 7.4 in water; | |
| | | pH 7.4 in water | |
| Anhydride | TA-NH₂ | CHCl3 or DMSO; | Amide |
| | TA-OH | 1.5:1 Base:PS-Y, | Ester |
| | TA-SH | CHCl₃; | Thioester |
| | | 1.5:1 Base:PS-Y, | |
| | | CHCl₃ | |
| Thiol | TA-SH | Oxidant CHCl₃ | Disulfide |

*Opt = optional;
NHS = N-hydroxy succinimide;
ITC = isothiocycanate;
IC = isocyanate

### EXAMPLES

The following examples are provided to illustrate various embodiments of systems, processes, compositions, applications and materials of the present inventions. These examples are for illustrative purposes, may be prophetic, and should not be viewed as, and do not otherwise limit the scope of the present inventions.

### Example 1

IR700 DX covalently attached to a small nanostructure (less than or equal to 25 nm in hydrodynamic diameter).

A dosing of less than or equal to 450 mg/kg particle in humans.

A therapeutic dosage of light administered that does not exceed 85% of the power that would yield thermal breakdown.

The use of IR700 DX as both a therapeutic or imaging agent.

Optionally attaching secondary imaging agents that may be fluorophores or radioagents (e.g. technetium).

Where a peptide, protein, antibody, small molecule, or otherwise any other entity that would act as a targeting agent to cardiac tissue is attached to the nanostructure. Preferably, the TA is one of the CTPs of Table 1.

Use of linear and multi-armed PEGs, but may also include any structure or material that fulfills the less than or equal to 25 nm hydrodynamic diameter feature (e.g. polyamido amine dendrimers, PAMAM).

### Example 2

Use of linear PEG, in the embodiment of Example 1. Other structures such as any structure or material that fulfills the less than or equal to 25 nm hydrodynamic diameter feature (e.g. polyamido amine dendrimers, PAMAM) may be used.

### Example 3

Use of multi-arm PEGs, for the embodiment of Example 1. Other structures, such as any structure or material that fulfills the less than or equal to 25 nm hydrodynamic diameter feature (e.g. polyamido amine dendrimers, PAMAM).

### Example 4

A method of forming an IR700-NP-CTP nanocomposition is to attach the IR700 to the NP, in the required ratio (e.g., 1-3 per NP) and to then attach a linker to the IR700 that have been attached to the NP. The CTP is then attached to this linker, as well as potentially other arms of the NP.

### Example 5

A PS-NP-TA nanocomposition, where PS is a phthalocyanine dye and the NP is 8PEG, 8PEGA, or 8PEGMAL and combinations of these, and the TA is a CTP.

### Example 6

A PS-NP-TA nanocomposition, where PS is a phthalocyanine dye and the NP is 8PEG, 8PEGA, or 8PEGMAL and combinations of these, and the TA is a CTP. The nanocomposition having a hydrodynamic diameter (e.g., size) of 25 nm and less, a hydrodynamic diameter of 10 nm and less, and having a hydrodynamic diameter of from about 30 nm to about 5 nm, and having a hydrodynamic diameter of from about 20 nm to about 5 nm, and being 20 kilodaltons (kDa) and greater, that are 40 kDa and greater, and that are from about 15 kDa to about 50 kDa, and that are about 5 kDa to about 100 kDa.

### Example 7

A PS-NP-TA nanocomposition, where PS is IR700 and the NP is 8PEG, 8PEGA, or 8PEGMAL and combinations of these, and the TA is one or more of SEQ ID NO: 1, SEQ ID NO: 2, SEQ ID NO: 3, SEQ ID NO: 4, SEQ ID NO: 37, SEQ ID NO: 38, SEQ ID NO: 39, SEQ ID NO: 40.

### Example 8

A PS-NP-TA nanocomposition, where PS is IR700 and the NP is 8PEG, 8PEGA, or 8PEGMAL and combinations of these, and the TA is one or more of SEQ ID NO: 1, SEQ ID NO: 2, SEQ ID NO: 3, SEQ ID NO: 4, SEQ ID NO: 37, SEQ ID NO: 38, SEQ ID NO: 39, SEQ ID NO: 40. The nanocomposition having a hydrodynamic diameter (e.g., size) of 25 nm and less, a hydrodynamic diameter of 10 nm and less, and having a hydrodynamic diameter of from about 30 nm to about 5 nm, and having a hydrodynamic diameter of from about 20 nm to about 5 nm, and being 20 kilodaltons (kDa) and greater, that are 40 kDa and greater, and that are from about 15 kDa to about 50 kDa, and that are about 5 kDa to about 100 kDa.

### Example 9

A PS-NP-TA nanocomposition, where PS is IR700 and the NP is 8PEG, 8PEGA, or 8PEGMAL and combinations of these, and the TA is at least one or more of SEQ ID NO: 1 to SEQ ID NO: 48.

### Example 10

A PS-NP-TA nanocomposition, where PS is IR700 and the NP is 8PEG, 8PEGA, or 8PEGMAL and combinations of these, and the TA is at least one or more of SEQ ID NO: 1 to SEQ ID NO: 48. The nanocomposition having a hydrodynamic diameter (e.g., size) of 25 nm and less, a hydrodynamic diameter of 10 nm and less, and having a hydrodynamic diameter of from about 30 nm to about 5 nm, and having a hydrodynamic diameter of from about 20 nm to about 5 nm, and being 20 kilodaltons (kDa) and greater, that are 40 kDa and greater, and that are from about 15 kDa to about 50 kDa, and that are about 5 kDa to about 100 kDa.

### Example 11

The embodiments of Examples 1 to 10, in which the NP is a 6PEG, 6PEGA, or 6PEGMAL and combinations of these instead of 8PEG.

### Example 12

The embodiments of Examples 1 to 10, in which the NP is a 4PEG, 4PEGA, or 4PEGMAL and combinations of these, instead of 8PEG.

### Example 13

The embodiments of Examples 1 to 10, in which the nanocomposition has one or more of the following parameters: from 1 to 2 PSs per NP; from 3 to 5 TAs per NP; the ratio of TA to PS is 2.5 to 1 and greater; the ratio of TA to PS is 3 to 1 and greater; the ratio of TA to PS is 5 to 1 and greater; having 1 free arm; having 2 free arms; having 3 free arms; and having 4 free arms.

### Headings and Embodiments

It should be understood that the use of headings in this specification is for the purpose of clarity, and is not limiting in any way. Thus, the processes and disclosures described under a heading should be read in context with the entirely of this specification, including the various examples. The use of headings in this specification should not limit the scope of protection afford the present inventions.

It is noted that there is no requirement to provide or address the theory underlying the novel and groundbreaking processes, materials, performance or other beneficial features and properties that are the subject of, or associated with, embodiments of the present inventions. Nevertheless, various theories are provided in this specification to further advance the art in this area. The theories put forth in this specification, and unless expressly stated otherwise, in no way limit, restrict or narrow the scope of protection to be afforded the claimed inventions. These theories many not be required or practiced to utilize the present inventions. It is further understood that the present inventions may lead to new, and heretofore unknown theories to explain the function-features of embodiments of the methods, articles, materials, devices and system of the present inventions; and such later developed theories shall not limit the scope of protection afforded the present inventions.

The various embodiments of systems, therapies, processes, compositions, applications, and materials set forth in this specification may be used for various other fields and for various other activities, uses and embodiments. Additionally, these embodiments, for example, may be used with: existing systems, therapies, processes, compositions, applications, and materials; may be used with systems, therapies, processes, compositions, applications, and materials that may be developed in the future; and with systems, therapies, processes, compositions, applications, and materials that may be modified, in-part, based on the teachings of this specification. Further, the various embodiments and examples set forth in this specification may be used with each other, in whole or in part, and in different and various combinations. Thus, for example, the configurations provided in the various embodiments of this specification may be used with each other. For example, the components of an embodiment having A, A′ and B and the components of an embodiment having A″, C and D can be used with each other in various combination, e.g., A, C, D, and A. A″ C and D, etc., in accordance with the teaching of this specification. The scope of protection afforded the present inventions should not be limited to a particular embodiment, example, configuration or arrangement that is set forth in a particular embodiment, example, or in an embodiment in a particular figure.

The invention may be embodied in other forms than those specifically disclosed herein without departing from its spirit or essential characteristics. The described embodiments are to be considered in all respects only as illustrative and not restrictive.

1. A nanocomposition comprising:
a. a photosensitizer (PS), wherein the photosensitizer is a phthalocyanine dye;
b. a nanoparticle (NP); wherein the nanoparticle is 8PEG; and,
c. a targeting agent (TA), wherein the targeting agent is a cardiac targeting peptide (CTP).

2. The nanocomposition of claim 1, wherein the nanocomposition is configured to provide a photodynamic therapy for a cardiac indication.

3. The nanocomposition of claim 2, wherein the PS is IR700; the 8PEG is selected from the group constituting of 8PEGA and 8PEGMAL, and the CTP is one or more of SEQ ID NO: 1, SEQ ID 2, SEQ ID NO: 37 and SEQ ID NO: 38.

4. The nanocomposition of claim 2, wherein the PS is IR700; the 8PEG is selected from the group constituting of 8PEGA and 8PEGMAL, and the CTP is one or more of SEQ ID NO: 1 to SEQ ID 48.

5. The nanocomposition of claim 2, wherein the PS is IR700; the 8PEG is selected from the group constituting of 8PEGA and 8PEGMAL, and the CTP is one or more of SEQ ID NO: 1 to SEQ ID 48; and having 3 and less PS per NP.

6. A nanocomposition, for use in treating a cardiac condition, the nanocomposition comprising:
a. a photosensitizer (PS), wherein the photosensitizer is a phthalocyanine dye;
b. a nanoparticle (NP); wherein the nanoparticle is selected from the group of 8PEG, 8PEGA and 8PEGMAL; and,
c. a targeting agent (TA), wherein the targeting agent is a cardiac targeting peptide (CTP);
d. wherein the nanocomposition is configured for providing a photodynamic therapy for the cardiac condition.

7. The nanocomposition of claim 6, wherein the CTP is one or more of SEQ ID NO: 1, SEQ ID 2, SEQ ID NO: 37 and SEQ ID NO: 38.

8. The nanocomposition of claim 6, wherein the CTP is one or more of SEQ ID NO: 1 to SEQ ID 48.

9. The nanocomposition of claim 6, wherein the nanocomposition has less than 3 PS per NP.

10. The nanocomposition of claim 6, wherein the cardiac condition is an arrhythmia.

11. The nanocomposition of claim 6, wherein the cardiac condition is selected from the group consisting of atrial fibrillation, premature atrial contractions, wandering atrial pacemaker, multifocal atrial tachycardia, atrial flutter, supraventricular tachycardia, tachycardia, junctional rhythm, junctional tachycardia, premature junctional contraction, and premature ventricular contractions.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A composition for use in treating a cardiac condition using a photodynamic therapy, the composition comprising:
a. a photosensitizer (PS), wherein the photosensitizer is a phthalocyanine dye;
b. a core molecule; and,
c. a targeting agent (TA), wherein the TA is specific to cardiac tissue.

24. (canceled)

25. (canceled)

26. (canceled)

27. The composition of claim 23, wherein the PS, TA or both are directly attached to the core molecule.

28. The composition of claim 27, wherein the direct attachment is a covalent bond.

29. The composition of claim 23, wherein the PS, TA or both are attached to the core by a linking moiety.

30. (canceled)

31. The composition of claim 23, wherein the TA is attached to the PS.

32-63. (canceled)
